(19) United States
(12) Patent Application Publication
(10) Pub. No.: US 2024/0376501 A1
(43) Pub. Date: Nov. 14, 2024

(54) Engineered Living Materials

(71) Applicant: UCL Business Ltd, London Greater London (GB)

(72) Inventors: Prantar Mahanta TAMULI, London Greater London (GB); Anete Krista SALMANE, London Greater London (GB); Brenda PARKER, London Greater London (GB); Marcos CRUZ, London Greater London (GB); Nina JOTANOVIC, London Greater London (GB)

(73) Assignee: UCL Business Ltd, London Greater London (GB)

(21) Appl. No.: 18/692,743

(22) PCT Filed: Sep. 16, 2022
(86) PCT No.: PCT/GB2022/052360

§ 371 Date: Mar. 15, 2024

(30) Foreign Application Priority Data
Sep. 17, 2021 (GB) GB 2113331.9

(51) Int. Cl. C12P 3/00; C12N 1/20; C12N 9/88

(52) U.S. Cl. CPC C12P 3/00; C12N 1/20; C12N 9/88; C12Y 402/01001

(57) ABSTRACT

The inventions related to a method of producing a biomineralized material comprising calcium carbonate-bonded aggregate, the method comprising: culturing a photosynthetic microorganism in a hydrogel matrix, wherein the microorganism releases extracellular carbonic anhydrase into the hydrogel matrix, and wherein the hydrogel matrix comprises: i) a hydrogel; ii) an aggregate material; iii) growth media; and iv) calcium chloride (CaCl₂), wherein the extracellular carbonic anhydrase converts the calcium chloride to calcium carbonate precipitate thereby bonding the aggregate material to form the biomineralized material; and associated materials, compositions and uses.

Contemporary advances in fields like biotechnology, material science and artificial intelligence are opening pathways to new transdisciplinary domains of research that were not possible before. Engineered Living Materials (ELMs) is one such emerging and rapidly developing domains. ELMs are smart biologically augmented materials whose assembly and properties are governed by living cells. Historically materials in human civilization have been looked at as pre-existing inanimate elements that can be extracted, reshaped and re-combined through purely physical means such as heating or pressurizing. Though these processes have achieved tremendous feats to produce the incredible assortment of materials that exists today, this approach primarily ends in results that have fixed physical and chemical properties. Moreover, they are often a one-way energy transfer mechanism and therefore unsustainable considering the global environmental challenges that we face today. On the contrary, all living systems operate through a parallel mechanism where biological materials-proteins and tissues, needed for their sustenance are grown or autonomously assembled bottom up. Biological materials are hierarchically graded and therefore extremely efficient. They are responsive to the environmental conditions and therefore inherently flexible in their properties, or self-healing and regenerative. And these materials fit within larger cycles of energy flows that make them environmentally sustainable. The field of Engineered Living Materials is driven by the aim to embed these complex properties that are present only in living systems till now into functional designed materials.

One approach to produce ELMs is effectively top-down where cells and biofilms are suspended in matrixes or in scaffolds to grow and produce desirable outcomes. This approach relies on tissue engineering techniques and produces ELMs that are artificially generated living composites containing biological and non-biological elements.

Key limitations and challenges exist across the ELM domains at present. The first challenge relates to scaling up since most of the bottom-up approaches work at microscale structuring. In certain fields like micro-electronics and robotics, scale might not be an issue however in other industries such as construction it is of vital importance. The second challenge is time span of production and growth and is directly related to the first one. Since biological process are slow, these ELMs may not be grown in a commercially viable timeline. The third challenge is maintaining long term viability of the cells/organisms. ELMs based on chemotrophic systems need to be maintained in the right environmental conditions and continuously fed with appropriate nutrients. For systems that rely on genetically modified organisms, biocontainment is an additional challenge during applications.

Although ELMs that operate with wild-type organisms do not face that challenge, natural living systems often operate within a complex network/consortium of other cells and microorganisms.

International patent application publication number WO2020/180914 describes an ELM production process where cyanobacterial cells are cultured and added to a mixture of hydrogel and aggregates. The mixture is maintained in a simulated 50%-100% humidity conditions to keep the metabolism of the cells intact and form mineral precipitates. However, once mixed the cells do not grow and their mass decreases, with just 9-14% active cells after 30 days under simulated conditions. Whilst material is produced, it is effectively not living material for an extended period of time and it does not continuously sequester carbon after the material formation. Furthermore, the material cannot operate at temperatures greater than 40° C., because the gelatine matrix can melt. The production can also be costly with a requirement for large bioreactors and aggregate sterilisation facilities.

New tool sets and techniques need to be developed for the study, production, and application of ELMs.

According to a first aspect of the present invention, there is provided a method of producing a biomineralized material comprising calcium carbonate-bonded aggregate, the method comprising:
culturing a photosynthetic microorganism in a hydrogel matrix, wherein the microorganism releases extracellular carbonic anhydrase into the hydrogel matrix, and
wherein the hydrogel matrix comprises:
i) a hydrogel;
ii) an aggregate material;
iii) growth media; and
iv) calcium chloride (CaCl₂)),
wherein the extracellular carbonic anhydrase converts the calcium chloride to calcium carbonate precipitate thereby bonding the aggregate material to form the biomineralized material.

This method of bio-manufacturing provides many advantages. The biomineralized material (which may also be termed “Engineered Living Material” (ELM)) has the potential to reduce the carbon footprint of construction and fabrication without the need for cement. The microorganism converts dissolved carbon dioxide into calcium carbonate, which acts as the binder for the aggregates. It can be grown within a span of weeks under natural room temperature and conditions. Advantageously, the resulting biomineralized material allows for a long-term viability of the microorganism, such as a cyanobacteria, or spore-like cells of the microorganism. Therefore, material can withstand periods of desiccation and the mineralizing process can be re-activated with addition of more growth media and calcium, which means that the biomineralized material can be self-healing. Additionally, old biomineralized material samples can be used as an inoculum mixed with new aggregates and growth media to produce new batches of materials, i.e. the material is regenerative. The invention provides for a truly bottom-up method to create a scalable, regenerative, photosynthetically active, biomineralized engineered living material that surpasses limitations currently present in the domain of ELMs. An advantage of the material is that it can also maintain carbon sequestration from production through to post-production use with the ability to support extended periods of the microorganism's viability. The material is non-fired and can be adapted to be hard and brittle or malleable depending on the aggregate size and desired application. The material can be cast in a mould during production and/or it can be machined to shape. The produced material can be translucent, and this translucency provides benefits not only the technical production and continued viability/regenerative ability, but also provides unique aesthetic qualities for several applications.

The most significant impact of this biomineralized material can be in the construction industry as replacement for cement-based materials. In particular, it can have a significant application as a construction material—it may be installed as a photosynthetic façade cladding material or could be used as in-fill material to replace traditional bricks depending on structural loading conditions. The material can be formed as a product range of blocks, tiles, and panels. UN estimations show that 2.5 billion more buildings will be constructed by 2050 to sustain the growing world population. Manufacturing of cement which is used as a primary material for construction today is known to be one of the biggest sources of carbon dioxide emissions cumulating to 5-6% of total pollution. Given the global climate emergency, this living biomineralized material offers a possibility for carbon neutral (even carbon negative) construction for the coming decades. It can also be applied as a material for eco-friendly products in various other industries such as sustainable interior design material, such as lighting elements, decorative fixtures and furniture.

### The Aggregate Material

The aggregate material may be sufficiently translucent as to allow photosynthetic growth of a photosynthetic microorganism, such as cyanobacteria, within the biomineralized material. In a preferred embodiment, the aggregate material is transparent or translucent. In one embodiment, the aggregate material is not opaque. The aggregate material may have a total transmittance value that is substantially the same as, or greater than, amorphous silica. Alternatively, the aggregate material may have a total transmittance value that is substantially the same as, or greater than, amorphous silica mixed with activated carbon in a ratio of 3:1. In one embodiment the aggregate material may have a total transmittance value that is within 20%+/−of the total transmittance of amorphous silica. In one embodiment the aggregate material may have a total transmittance value that is within 10%+/−of the total transmittance of amorphous silica. In one embodiment the aggregate material may have a total transmittance value that is within 5%+/−of the total transmittance of amorphous silica. The skilled person will recognise that the thickness of the material to be produced may determine the level of translucency required for the aggregate in order to allow photosynthesis. In particular, the translucency may be sufficient to allow photosynthesis. The production can also be supported by higher light intensities and/or integrated lighting.

The translucency or transparency may be determined when the aggregate material is wet or dry. The skilled person will recognise that inside the hydrogel the aggregates are wet and aggregate components such as amorphous silica can be more transparent when wet and translucent when dry. The translucency or transparency of an aggregate may be sufficient for photosynthesis when it is wet within the hydrogel.

The skilled person will recognise that the zone in which photosynthesis can occur is termed the euphotic zone (Zeu). By definition, the euphotic zone extends from the surface to the depth at which 1 percent of the surface light intensity can be detected.

As an example, the minimum light intensity for Cyanobacteria growth is about 36 μmol m⁻²s⁻¹. Since the transmittance is dependent on the depth of the material, for example as it is cast, the aggregate must be able to have a transmittance such that at the required functional depth it is able to provide 1% of the surface light conditions for growth of a photosynthetic microorganism such as cyanobacteria, such as at least 36 μmol m⁻²s⁻¹.

In one embodiment, the aggregate material comprises or consists of amorphous and/or crystalline material. The aggregate material may comprise a material selected from amorphous silica (SiO₂), crystalline quartz silica, glass particles (e.g. recycled glass particles), transparent ceramics, polyacrylate and glass fibres; or combinations thereof.

In one embodiment, the aggregate material comprises amorphous silica (SiO₂). The aggregate material may further comprise activated carbon pellets and/or zeolite. In one embodiment, the aggregate material comprises or consists of activated carbon pellets and amorphous silica (SiO₂).

The aggregate material may comprise nano-fibres, such as cellulose nano-fibres. In one embodiment, the aggregate material comprises or consists of the porous particulate, such as activated carbon pellets, amorphous silica (SiO₂), and nano-fibres, such as cellulose nano-fibres.

In one embodiment, the aggregate material comprises or consists of the porous particulate, such as activated carbon pellets, and amorphous silica (SiO₂) in a ratio of 1:3. In another embodiment, the aggregate material comprises or consists of the porous particulate, such as activated carbon pellets, and amorphous silica (SiO₂) in a ratio of between 1:2 and 1:4. The aggregate material may be a translucent solid particulate and/or translucent fibrous material. A combination of different aggregates may be provided.

The aggregate material may not comprise sand or gravel. The aggregate material may not comprise more than 10% sand and/or gravel. In another embodiment, the aggregate material may not comprise more than 5% sand and/or gravel.

Advantageously, the use of translucent aggregate material allows for extended viability of photosynthetic microorganisms, such as a cyanobacteria. Translucent aggregate material allows for the resulting biomineralized material to be translucent and colour changing. For example, the biomineralized material can dynamically change colours between yellow, green, and blue depending on light exposure and nutrient conditions.

In addition to translucency or transparency properties, the skilled person will appreciate that the aggregate material may also be chosen based on the desired physical properties of the resulting biomineralized material. For example quartz has a higher compressive strength than amorphous silica and glass fibres. However, glass fibres can provide greater tensile strength to the material. Different forms of aggregates may be combined in various ratios to create desired properties.

The aggregate may comprise or consist of a particulate material having a micro-porous surface. The micro-surface capacity of the aggregate material may be at least 750 m²/g. In another embodiment, the micro-surface capacity of the aggregate material may be at least 1000 m²/g. In another embodiment, the micro-surface capacity of the aggregate material may be at least 1300 m²/g. In another embodiment, the micro-surface capacity of the aggregate material may be between 750 m²/g and about 3000 m²/g. In another embodiment, the micro-surface capacity of the aggregate material may be between 750 m²/g and about 1500 m²/g. Where the aggregate comprises two or more different particle types, the micro-surface capacity of the aggregate material may refer to the average micro-surface capacity of the combined aggregate material. For example, if the aggregate comprises SiO₂ having a micro-surface capacity of 750 m²/g and comprises activated carbon pellets having a micro-surface capacity of about 3000 m²/g, the average micro-surface capacity of the aggregate mixture combined in a 3:1 ratio may be about 1350 m²/gm.

The activated carbon pellets may not be powder, such as may not be 1 mm in size or less. The activated carbon pellets may be 2 mm or more in size, preferably 3 mm or more. The activated carbon pellets may be about 2-6 mm in size, preferably 3-5 mm in size. Reference to the pellet size is intended to refer to an average size in a population of pellets.

In one embodiment, the aggregate comprises a hydroscopic material, preferably a translucent hygroscopic material. In one embodiment, the aggregate comprises a hygroscopic, translucent and micro-porous material.

Advantageously, aggregates with a high micro-porous surface capacity such as activated carbon pellets and SiO₂ exhibit inherent binding tendency with biofilms. The micro-porous surface allows cyanobacteria trichomes to interweave through the porosity of adjacent particles resulting in a continuous mat. Activated carbon pellets have a micro-surface capacity of 3000 m²/g however they are opaque and not ideal for light transmission. Therefore, they may be more useful as a minor component. SiO₂ has a lower micro surface capacity of 800 m²/g but it is translucent when dry and transparent when wet. Silica is also naturally hydroscopic and widely used as a desiccant because of its ability to adsorb moisture from air. Also, compared to river sand which is becoming a scare resource for construction, silicon dioxide is known as the second most abundant mineral on Earth and an great untapped resource potential. Thus, silica is an ideal aggregate for the ELM.

In one embodiment, the aggregate comprises or consists of particles about 5 mm in diameter. In another embodiment, the aggregate comprises or consists of particles between about 2 mm and about 8 mm in diameter. Reference to the diameter of a particle is intended to refer to the largest dimension of the particle, and an average diameter of a population of aggregate particles. In one embodiment, the aggregate comprises or consists of particles about 3-5 mm in diameter. The aggregate may not be powder, such as may not be 1 mm in size or less. The aggregate may be 2 mm or more in size, or 3 mm or more. The aggregate may be about 2-6 mm in size, preferably 2-5 mm in size.

It is understood that the skilled person may choose different particle sizes depending on the application of the resulting material. For example, higher diameter particles of greater than 3 mm, such as 3-5 mm, provide a more brittle material, and lower diameter particles, for example 3 mm or less, can provide more malleable material.

### The Microorganism

The microorganism may exhibit (i.e. be capable of) one or more of gliding motility, phototactic response and chemotactic response. In one embodiment, the microorganism exhibits (i.e. is capable of) gliding motility, and phototactic response and/or chemotactic response. In another embodiment, the microorganism exhibits (i.e. is capable of) gliding motility, phototactic response and chemotactic response. The chemotactic response may be in response to one or more, or all of CO₂, O₂ and HCO₃ ion concentrations. The phototactic response may be in response to directional light.

In a preferred embodiment, the microorganism is photosynthetic, for example the microorganism may comprise cyanobacteria.

Use of photosynthetic microorganisms allow to minimise the input of high-cost carbon sources used to maintain heterotrophic microorganisms.

Advantageously, the provision of a microorganism, such as a filamentous cyanobacteria, having gliding motility, and phototactic response and/or chemotactic response allows the control of growth direction, and the ability to program the mineralization for designed applications.

In one embodiment, the microorganism is a bacteria, such as a bacteria capable of biofilm formation. The microorganism may comprise or consist of a cyanobacteria. Preferably, the cyanobacteria is a filamentous cyanobacteria.

Preferably, the microorganism, such as a cyanobacteria, can form spores or spore-like cells. In an embodiment wherein the microorganism is a cyanobacteria, the cyanobacteria may be capable of forming dormant cells (or otherwise termed “spore-like cells”), which may include akinetes and/or heterocysts (i.e. nitrogen fixing cells).

Advantageously, the ability to form spores or spore-like cells allows for continuous rejuvenation of the biomineralized material. For example, the material may be applied in environments where continuous reproductive cell viability could be difficult to maintain. However, the ability to form spores or spore-like cells allows for changing conditions, periods of reproductive growth and periods of dormancy, such as dry and hot conditions.

The microorganism may comprise a microorganism of the Nostocales order of cyanobacteria, such as Nostoc spp. The microorganism may comprise a microorganism of the family of Oscillatoriaceae (a family of cyanobacteria taxonomy). The microorganism may be selected from Oscillatoria spp., Spirulina spp. Nostoc spp. and Tolypothrix spp; or combinations thereof. The microorganism may be selected from Oscillatoria spp., Spirulina spp. and Tolypothrix spp; or combinations thereof. The microorganism may comprise Oscillatoria spp. and/or Spirulina spp. The Oscillatoria spp. may be Oscillatoria animalis. The Spirulina spp. may be Spirulina platensis. In a preferred embodiment, the microorganism comprises or consists of Oscillatoria animalis.

Advantageously, many species in the Oscillatoriacles order of cyanobacteria are naturally evolved to survive harsh terrestrial conditions like deserts and rocks surfaces, which can make the ideal for use in the material of the invention. For example, Oscillatoria animalis has been demonstrated herein to exhibit extremely long duration of sustained growth for over 180 days. A defining characteristic of the members of the Oscillatoriaceae family is the rotation of the filaments during the process of gliding. Therefore, the cyanobacteria, such as one of the Oscillatoriaceae family, may display rotation of its filament while gliding in pure methylcellulose (4%-15%) or methylcellulose-based hydrogel compositions with up to 1% sodium alginate.

In one embodiment a co-culture of two or more microorganisms are provided, such as two or more cyanobacteria species or strains.

Preferably the microorganism is a natural producer of extracellular carbonic anhydrase. However, the skilled person will appreciate that a microorganism may be modified to express extracellular carbonic anhydrase. In one embodiment, the microorganism is modified to produce carbonic anhydrase, for example by transformation with nucleic acid encoding a recombinant carbonic anhydrase.

The amount of microorganism provided may be a sufficient amount to provide precipitation of the calcium carbonate throughout the hydrogel to bond the aggregate material. In one embodiment, amount of microorganism provided is an amount sufficient to provide complete growth throughout the hydrogel within 21 days of mineralizing period. In one embodiment, amount of microorganism provided as an inoculum is an amount sufficient to cover at least 75% of the surface area of the hydrogel.

Preferably, the microorganism is provided as an inoculum and cultured to achieve active growth within the hydrogel. Such culturing may be otherwise known as a mineralizing period. The microorganism may not be fully cultured separately from the hydrogel and then mixed, where no further growth is encouraged or achieved.

An advantage of the method of the invention is that there is no need to culture the microorganism separately and then mix into the hydrogel composition. The hydrogel matrix along with the aggregates itself can act as a 3D growth scaffold for the microorganism. The microorganism, such as cyanobacteria, produces carbonic anhydrase as a by-product of its metabolic activity for growth which enables efficient biomineralization during the growth.

### The Hydrogel

In one embodiment the hydrogel is thixotropic. In particular, the hydrogel may be sufficiently viscous to maintain its 3D shape, but is capable of sufficient cyanobacteria fluidity under stress for allowing gliding motility of a microorganism, such as a filamentous cyanobacteria.

In one embodiment, the hydrogel comprises methylcellulose. In another embodiment, the hydrogel comprises or consist of methylcellulose and one or more other organic gel compounds. The hydrogel may comprise or consist of a majority (i.e. greater than 50% w/w) of methylcellulose, and one or more other organic gel compounds as a minor component(s) (i.e. less than 50% w/w in total).

The hydrogel may not comprise or consist of gelatine.

The one or more other organic gel compounds may comprise or consist of sodium alginate. The one or more other organic gel compounds may comprise or consist of sodium alginate, agar and carrageenan; or combinations thereof.

The methylcellulose and one or more other organic gel compounds may be in a ratio of between about 15:1 and 3:1. Alternatively, the methylcellulose and one or more other organic gel compounds may be in a ratio of between about 10:1 and 3:1. Further alternatively, the methylcellulose and one or more other organic gel compounds may be in a ratio of between about 8:1 and 3:1. In another embodiment, the methylcellulose and one or more other organic gel compounds is in a ratio of about 6:1.

In a preferred embodiment, the hydrogel comprises or consists of methylcellulose and sodium alginate. The methylcellulose and sodium alginate may be in a ratio of between about 15:1 and 3:1. Alternatively, the methylcellulose and sodium alginate may be in a ratio of between about 10:1 and 3:1. Further alternatively, the methylcellulose and sodium alginate may be in a ratio of between about 8:1 and 3:1. In a preferred embodiment, the methylcellulose and sodium alginate is in a ratio of about 6:1.

Methylcellulose gel is known to increase in viscosity at higher temperatures. The provision of a mixture of methylcellulose and sodium alginate, for example at a ratio of 6:1, advantageously provides a composition that is thermally stable, where sodium alginate has a low gel viscosity at higher temperatures contrary to methylcellulose.

The amount of gel forming compound, such as methylcellulose, that is provided in the growth media to form the hydrogel matrix may be an amount sufficient enough to form the hydrogel. The gel forming compound, such as methylcellulose, may be provided in the growth media in the amount of about 6% w/v.

The hydrogel may comprise methylcellulose at 4%-16% w/v. In another embodiment, the hydrogel may comprise methylcellulose at 4%-16% w/v and sodium alginate at 0.1-1% w/v. In another embodiment, the hydrogel may comprise methylcellulose at 4%-16% w/v and agar at 0.1-1% w/v. In another embodiment, the hydrogel may comprise methylcellulose at 4%-16% w/v and carrageenan at 0.1-1% w/v.

The hydrogel matrix may be shaped, for example by pouring the hydrogel composition into a mould prior to setting as a hydrogel (this may also be known herein as “casting”). In one embodiment, the hydrogel matrix is provided as a layer. The layer may be between 0.2 cm and 5 cm in thickness. The layer may be about 1 cm in thickness, for example for structural applications. The layer may be less than 1 cm in thickness. In one embodiment, the hydrogel matrix is shaped into bricks or tiles (e.g. in a rectangular prism shape). The surface of the hydrogel matrix may be non-uniform (i.e. non-planar), for example comprising one or more of ridges, flanges, projections, indentations, grooves, channels, bumps, or undulations (e.g. wave-like patterns).

In another embodiment, the hydrogel may be 3D printed to form the desired shape prior to the biomineralization to form the biomineralized material. In another embodiment, the biomineralized material may be shaped, for example by milling or carving, after the biomineralization process.

The 3D shape of the hydrogel matrix can translate into the eventual 3D shape of the biomineralized material. The provision of surface patterns can provide aesthetic forms to the biomineralized material. A benefit of a non-uniform/non-planar surface is that the resulting ridges, flanges, projections, grooves, channels, bumps, or undulations can provide a function, where the hydrogel matrix and microorganism are situated in areas of higher or lower light intensity depending on their location. For example, a groove, indentation, or channel can help to partially shade the hydrogel matrix to protect it from desiccation.

### Growth Media

The skilled person will recognise that the growth media may be any growth media that is suitable to at least support the viability, and preferably the growth, of the microorganism. In an embodiment wherein the microorganism is a cyanobacteria, the growth media may be BG11 media, or an equivalent thereof. Appropriate trace elements may be provided in the growth media.

### The Calcium Chloride

The skilled person will recognise that any source of calcium ions may be used. The calcium chloride may comprise or consist of calcium chloride dihydrate solution (CaCl₂·2H₂O). Alternatively, the calcium chloride may comprise anhydrous calcium chloride.

The calcium chloride may be provided at a concentration of between about 0.01M and about 1M. In another embodiment, the calcium chloride may be provided at a concentration of between about 0.1M and about 1M. In one embodiment, the calcium chloride is provided at a concentration of about 1M. In another embodiment, the calcium chloride is provided at a concentration of about 0.1M. In another embodiment, the calcium chloride is provided at a concentration of about 0.01M.

### Growth Conditions

In an embodiment wherein the microorganism is photosynthetic and/or phototactic, the mineralizing period with the microorganism in the hydrogel matrix may be in the presence of a light source. The light source may be ambient light, or directional light. The light source may be natural light (i.e. daylight) or artificial light. The light source may be of sufficient intensity and wavelength to promote photosynthesis and/or phototaxis. The skilled person will recognise that the light intensity may be adjusted in accordance with factors such as the thickness of the material, the translucency of the aggregate material and the type of microorganism. The light may be provided at an intensity sufficient to provide a euphotic zone (Zeu) for photosynthesis to occur throughout the material. The light may be provided at an intensity of between about 360 lux and 3200 lux. The light may be provided at an intensity of about 2200 lux. The light may be provided at an intensity of at least 100 μmol m⁻²s⁻¹. In another embodiment, the light may be provided at an intensity of at least 200 μmol m⁻²s⁻¹. In another embodiment, the light may be provided at an intensity of at least 300 μmol m² s⁻¹. In another embodiment, the light may be provided at an intensity of at least 400 μmol m⁻²s⁻¹. In another embodiment, the light may be provided at an intensity of between 200 μmol m⁻²s⁻¹ and 800 μmol m⁻²s⁻¹. In another embodiment, the light may be provided at an intensity of between 400 μmol m⁻²s⁻¹ and 800 μmol m⁻²s⁻¹. The light may be provided at an intensity of no more than 800 μmol m⁻²s⁻¹. The light intensity may be sufficient for growth of the microorganism, and for example may not exceed a level in which the microorganism growth is retarded, for example by too much heat energy from the light source or too much drying effect.

The wavelength and intensity of light can determine the growth and output material properties. Red wavelength (e.g. about 750 nm) can trigger the phototactic movement of the cyanobacteria which can be used to control the growth direction of the cyanobacteria inside the material. Therefore, the light may of red wavelength, such as between 620 and 750 nm (e.g. about 750 nm). Blue wavelength (e.g. about 450 nm) can trigger rapid cellular division and potentially carbonic anhydrase production which can help more biomineral formations. Therefore, the light may of blue wavelength, such as between 450 and 495 nm (e.g. about 450 nm). Additionally, high intensity of light 400-800 μmol m⁻²s⁻¹ can increase biomineral formation.

A mineralizing period for the microorganism may be considered a period in which the growth process of the microorganism coincides with a mineralization period in the material in the method. The mineralizing period may also be known as a culturing of the microorganism. This culturing may not be in isolation from the hydrogel matrix.

The mineralizing period of the microorganism may be at a temperature suitable for maintenance and/or growth of the microorganism. In one embodiment, the temperature is ambient room temperature (e.g. about 24° C.). In another embodiment, the temperature is ambient outdoor temperature. In another embodiment, the temperature is between about 4° C. and about 24° C. In another embodiment, the temperature is between about 14° C. and about 24° C. In a preferred embodiment, the temperature is between about 20° C. and about 25° C. for optimal for biomineral formation.

The mineralizing period of the microorganism may be over a period of at least 14 days. In one embodiment, the mineralizing period of the microorganism may be over a period of about 21 days. In another embodiment, the mineralizing period of the microorganism may be over a period of up to 3 weeks. The biomineralized material may be sufficiently formed after 21 days of mineralizing period with the microorganism. The biomineralized material may be sufficiently formed after 21-45 days of mineralizing period with the microorganism.

The microorganism may remain viable for at least 75 days, for example under ambient conditions. In another embodiment, the microorganism may remain viable for at least 180 days, for example under ambient conditions. In an embodiment wherein the microorganism is cyanobacteria, the cyanobacteria may remain viable in a reproductive cell state for at least 75 or 180 days under hydrated conditions. The viability may be determined by the ability of the microorganism to continue to grow in the presence of nutrients.

After desiccation of the biomineralized material conditions, the microorganism may remain viable via spores or spore-like cells, and can be capable of regeneration into a reproductive cell state after rehydration, for example up to 6 months after desiccation.

In one embodiment, the mineralizing period with the microorganism in the hydrogel matrix may not be under sterile conditions.

Advantageously, the present invention has been demonstrated to form the biomineralized material regardless of sterility (e.g. for the avoidance of contaminants). This allows for a more practical and less resource intensive production procedure.

### Other Aspects

According to another aspect of the present invention, there is provided a method of producing a biomineralized material comprising calcium carbonate-bonded aggregate, the method comprising:
culturing a microorganism in a hydrogel matrix, wherein the microorganism releases extracellular carbonic anhydrase into the hydrogel matrix, and exhibits gliding motility, and phototactic and chemotactic response, and
wherein the hydrogel matrix comprises:
i) a hydrogel comprising or consisting of methylcellulose and one or more other organic gel compounds;
ii) an aggregate material;
iii) growth media; and
iv) calcium chloride (CaCl₂)),
wherein the extracellular carbonic anhydrase converts the calcium chloride to calcium carbonate precipitate thereby bonding the aggregate material to form the biomineralized material.

According to another aspect of the present invention, there is provided a composition for producing a biomineralized material comprising calcium carbonate-bonded aggregate, the composition comprising a hydrogel matrix, wherein the hydrogel matrix comprises:
i) a hydrogel;
ii) an aggregate material;
iii) growth media;
iv) calcium chloride (CaCl₂)); and
v) a microorganism, wherein the microorganism is capable of expressing and releasing extracellular carbonic anhydrase into the hydrogel matrix.

According to another aspect of the present invention, there is provided a composition for producing a biomineralized material comprising calcium carbonate-bonded aggregate, the composition comprising a hydrogel matrix, wherein the hydrogel matrix comprises:
i) a hydrogel;
ii) an aggregate material;
iii) growth media;
iv) calcium chloride (CaCl₂)); and
v) a microorganism, wherein the microorganism is capable of expressing and releasing extracellular carbonic anhydrase into the hydrogel matrix.

According to another aspect of the present invention, there is provided a biomineralized material comprising:
i) a hydrogel or a biogenic mineral of a dried hydrogel
ii) calcium carbonate-bonded aggregate material;
iii) a filamentous cyanobacteria or spore-like cells thereof capable of forming reproductive cells of the filamentous cyanobacteria, wherein the filamentous cyanobacteria is capable of expressing extracellular carbonic anhydrase.

According to another aspect of the present invention, there is provided a biomineralized material produced according to the method of the invention herein.

The biomineralized material may further comprise one or more additional microorganisms that are different to the filamentous cyanobacteria of iii). The one or more additional microorganisms may be a different species of cyanobacteria.

The biomineralized material can advantageously provide a translucent regenerative/self-healing material, which and can maintain carbon sequestration in extended period of use. The material can be aesthetically and/or functionally shaped, for example by pre-shaping the hydrogel from which it is formed, or by machining to shape. The ongoing viability of the filamentous cyanobacteria can allow the material to dynamically change colours between yellow, green, and blue depending on light exposure and nutrient conditions.

According to another aspect of the present invention, there is provided the use of Oscillatoria spp. to form a biomineralized material, wherein the use is in a hydrogel with an aggregate material, growth media, and calcium chloride (CaCl₂)), to facilitate calcium carbonate precipitation and bonding of the aggregate thereby forming the biomineralized material,
optionally characterised in that the hydrogel comprises or consists of methylcellulose and sodium alginate.

According to another aspect of the present invention, there is provided the use of the biomineralized material according to the invention for cladding on a building.

### Definitions

The term “gliding motility” is herein intended to refer to a method of translocation used by microorganisms that is independent of propulsive structures such as flagella, pili, and fimbriae. Gliding allows microorganisms to travel along the surface of low aqueous films.

The term “phototactic response” is herein intended to refer to the action of a whole organism that moves towards or away from a stimulus of light. This is advantageous for phototrophic organisms as they can orient themselves most efficiently to receive light for photosynthesis.

The term “chemotactic response” is herein intended to refer to the movement of an organism in response to a chemical stimulus.

The term “translucent” is herein intended to refer to the ability to transmit light. Translucency may include optical transparency.

Transparency of a material is measured by its total transmittance. Total transmittance is the ratio of transmitted light to the incident light. There are two influencing factors; reflection and absorption. For example: Incident light=100%−(Absorption=−1%+Reflection=−5%)=Total Transmittance=94%.

Thixotropy, is understood to be a reversible behaviour of certain gels that liquefy when they are shaken, stirred, or otherwise disturbed and reset after being allowed to stand. Thixotropy can be determined by rheology measurements involving one or more shear, flow and oscillation tests known to the skilled person.

The skilled person will appreciate that the optional features of the first aspect, or any aspect or embodiment, of the invention can be applied to all aspects of the invention.

Embodiments of the invention will now be described, by way of example only, with reference to the following examples.

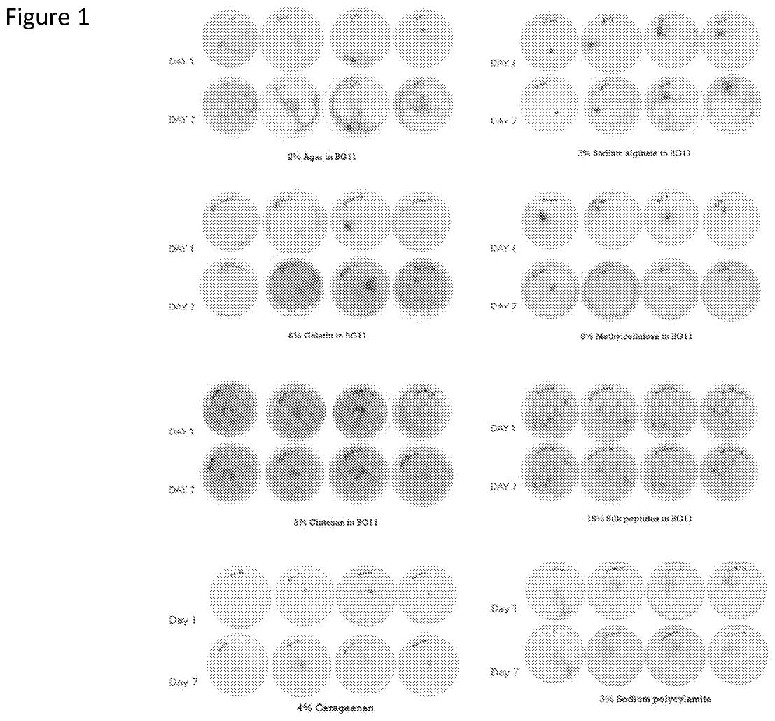

FIG. 1: Interaction of Oscillatoria animalis biofilm with different hydrogel compositions as mentioned over a 7-day period.

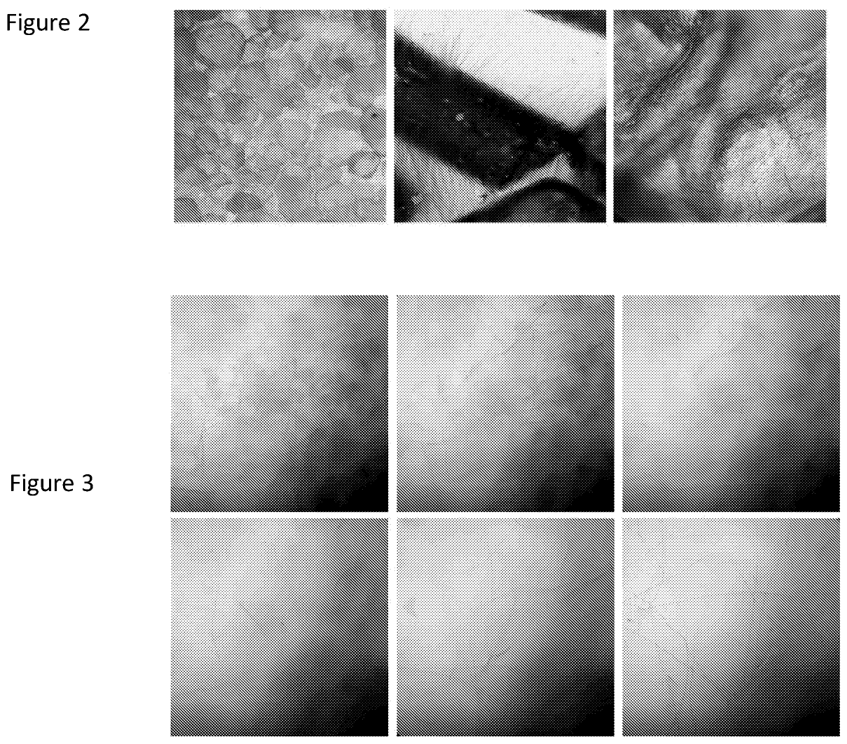

FIG. 2: Binding interaction of Oscillatoria animalis biofilm with high micro-porous aggregates like river sand (left), activated carbon (centre) and amorphous silica (right) at 400× magnification.

FIG. 3: 3-dimensional growth of Oscillatoria animalis trichomes as seen in the cross section of the Methylcellulose gel matrix at 400 magnification.

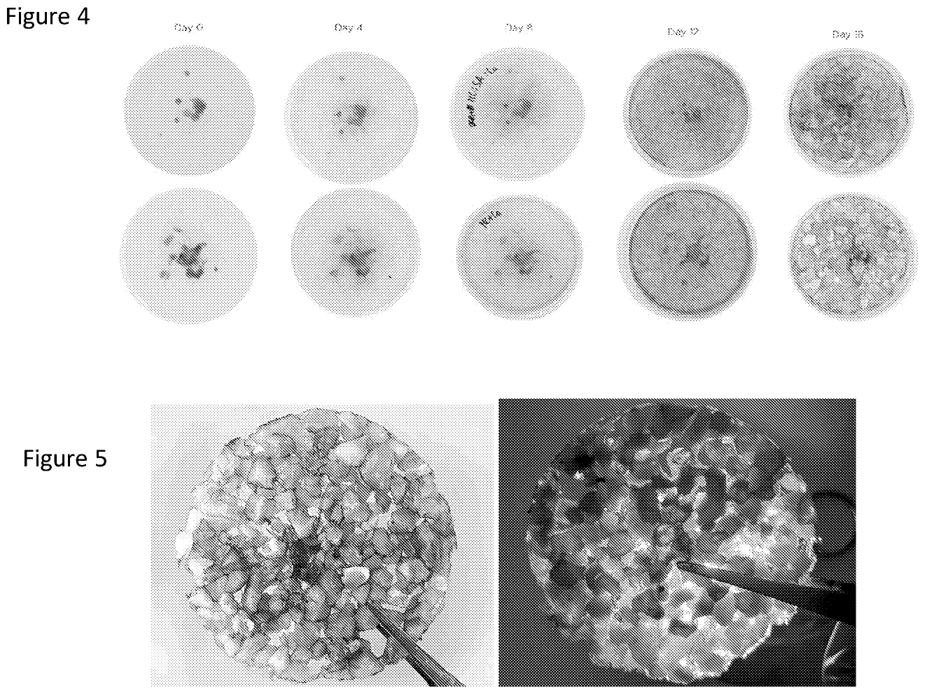

FIG. 4: Growth of photosynthetic engineered living material prototype over 16 day period. SiO₂ aggregates were suspended in 6:1 Methylcellulose-Sodium alginate hydrogel supplemented with 0.1M CaCl₂·2H₂O and inoculated with Oscillatoria animalis in a petri dish. The sample was completely grown in 12 days and dehydrated over 4 days.

FIG. 5: Demonstration of photosynthetic biomineralized engineered living material in day light (left) and under artificial light (right).

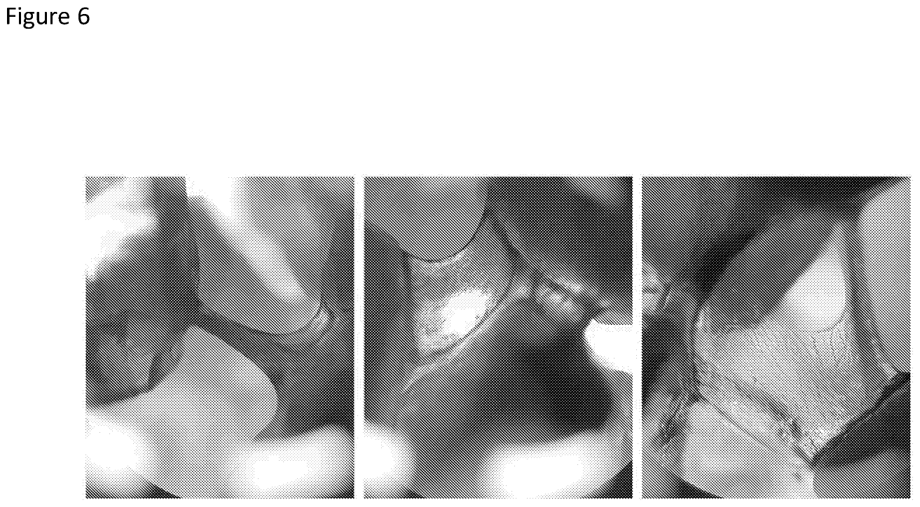

FIG. 6: Photosynthetically active cyanobacterial biofilm binding the SiO₂ aggregates in the matured ELM as seen at 400× magnification.

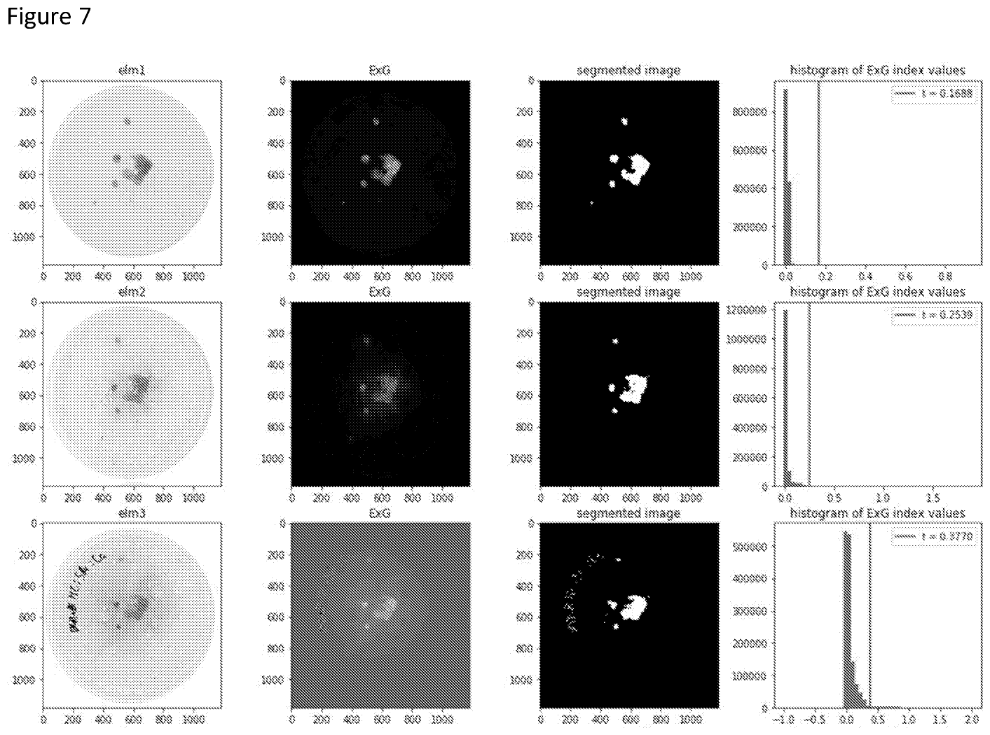

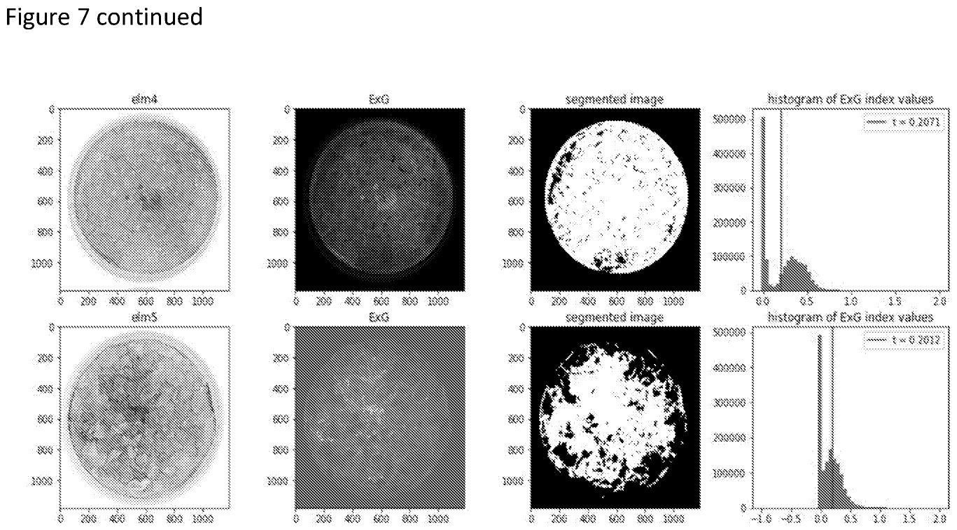

FIG. 7: Growth analysis with ExG mapping of PB-ELM. (In order from left column) Chronological images of growth, ii) ExG index identification, iii) pixel segmentation of ExG index, iv) histogram showing threshold value for segmentation.

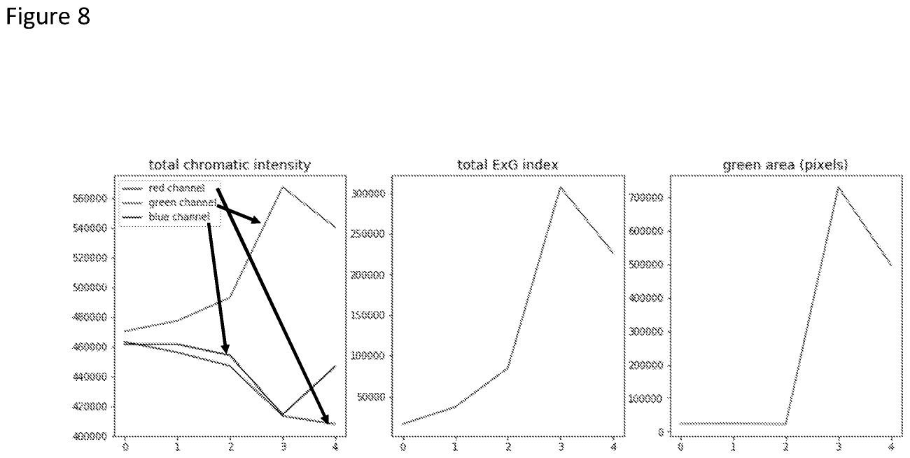

FIG. 8: Growth analysis maps of total chromatic intensity of RGB values (left), total ExG index (centre) and total green area (right).

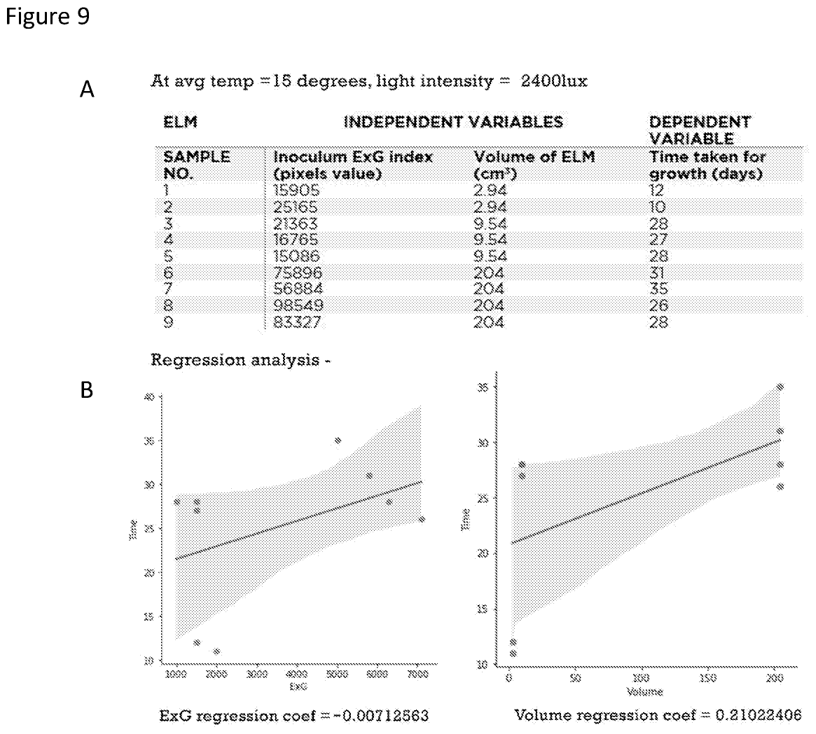

FIG. 9: A Data of ExG index, volume and days taken for growth of various ELM samples (above). B Linear regression plot of time (dependent value) against ExG and volume (independent values). R(ExG)=−0.007 and R(Volume)=0.210.

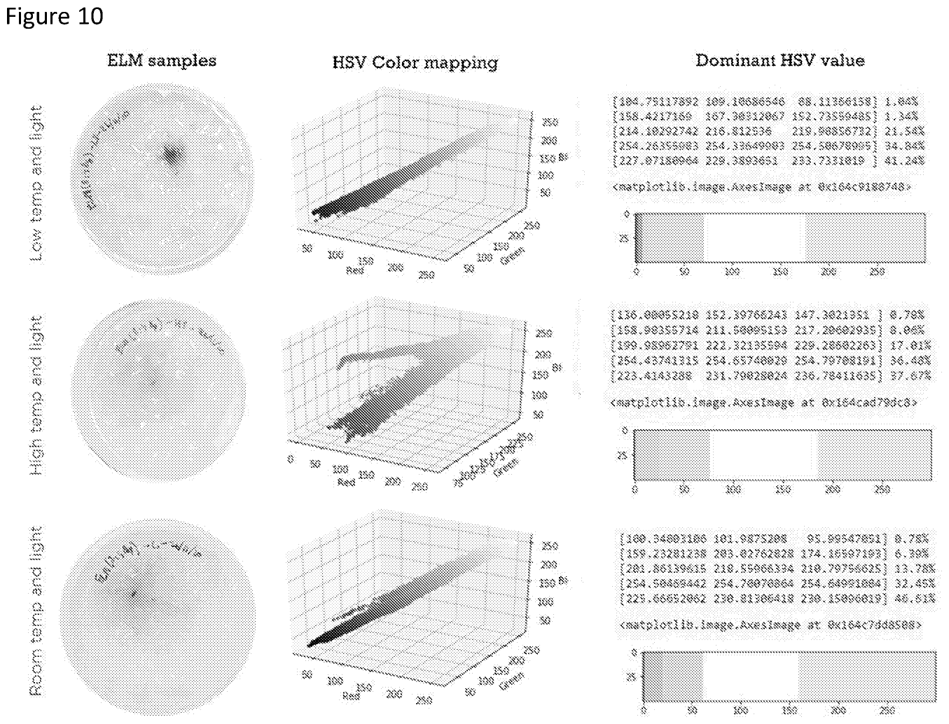

FIG. 10: Photosynthetic health identification of ELM by HSV analysis of samples grown in 4 degrees and 360 lux (top row), 24 degrees and 3200 lux (centre row) and at 14 degrees and 2200 lux (bottom row) over a period of 7 days.

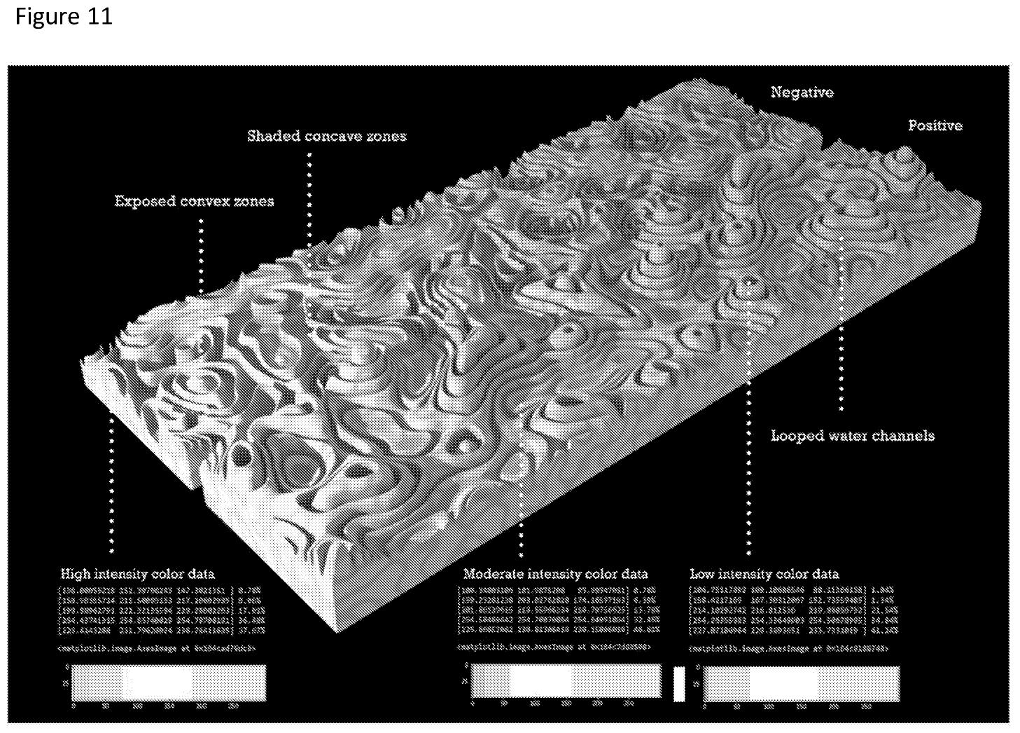

FIG. 11: Digital design of meso-scale prototype generated by mathematical modulo operator-based script and enhanced by HSV colour data input from PB-ELM analysis for simulated outcome visualization.

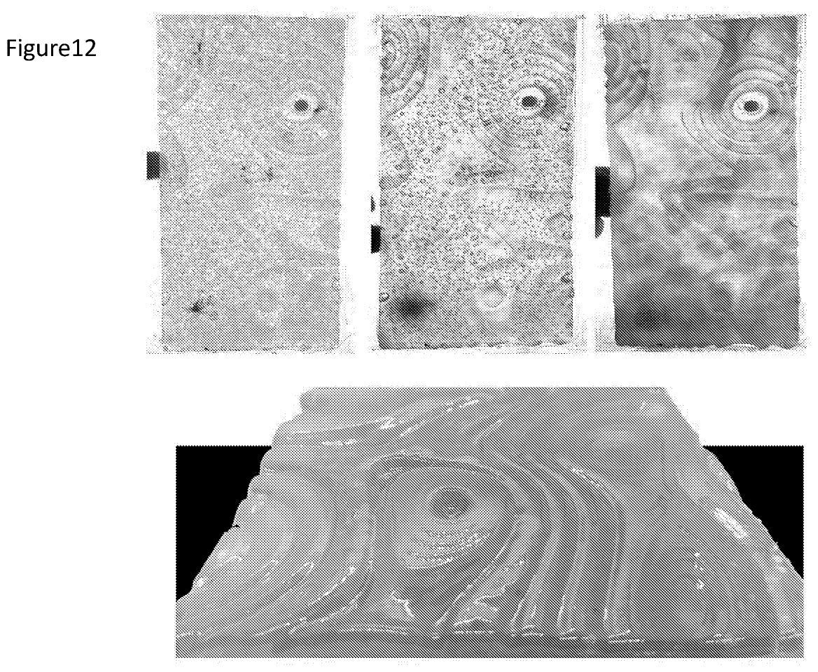

FIG. 12: Digital design were 3-d printed and vacuum formed to make transparent moulds. The moulds and incubated with the PB-ELM and grown over a period of 30-45 days. Growth stages at day 1, day 10 and day 20 (above) and close up of fully grown meso scale PB-ELM mould at day 34 (Below)

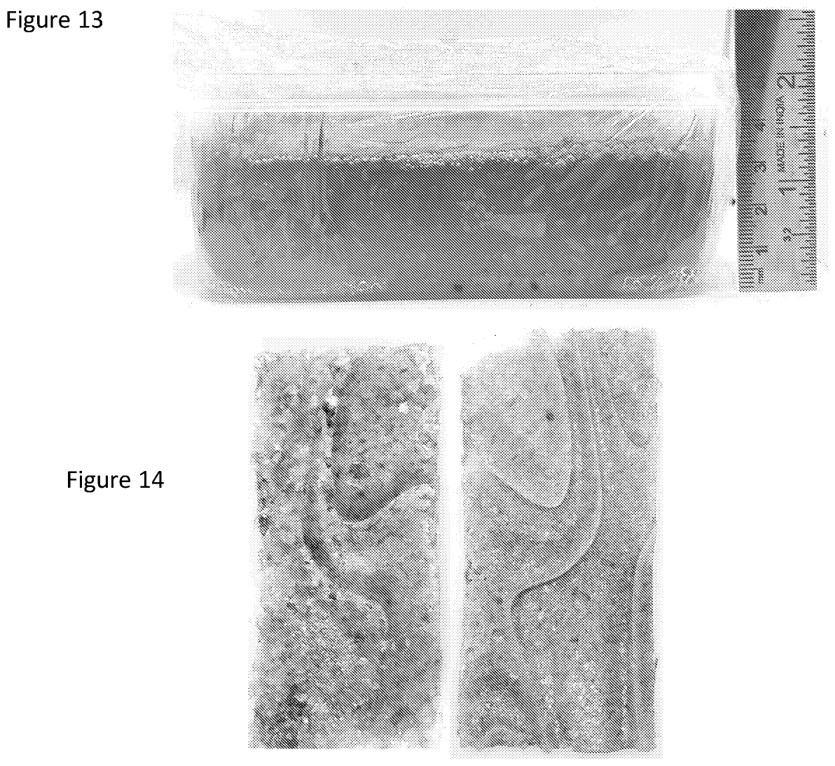

FIG. 13: Successful cyanobacteria growth up to 3.5 cm depth was observed in the meso-scale prototype.

FIG. 14: Demoulded photosynthetically active PB-ELM meso-scale panels/interlocking bricks of size 16×8×1.5 cm.

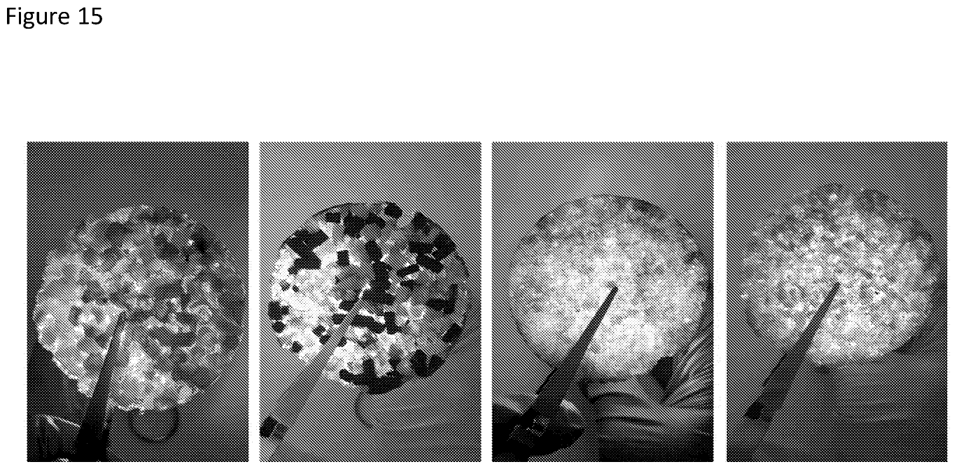

FIG. 15: Different PB-ELM versions with varied aggregate type and ratio-5 mm amorphous silica, 4:1 5 mm amorphous silica+5 mm activated carbon, 2 mm amorphous silica, 2:1 2 mm amorphous silica+amorphous silica powder.

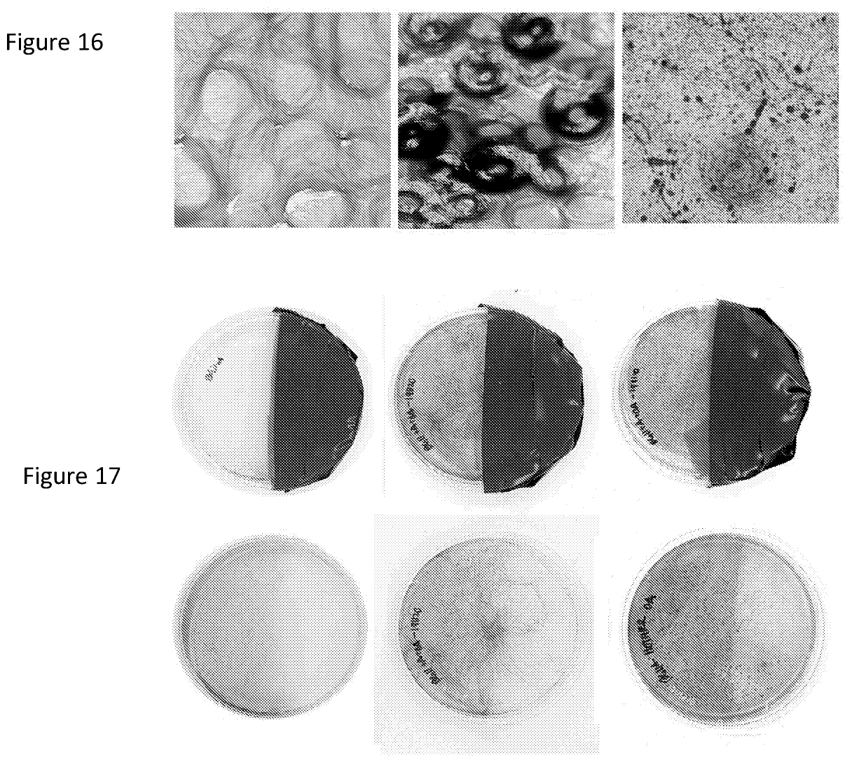

FIG. 16: Oscillatoria animalis typical biofilm pattern (left), dormant cell formation-dark thick-walled cells, observed under moisture deficient condition (centre) and isolated dormant cells seen as green spots in 6-month-old dehydrated hydrogel (right) at 400× magnification.

FIG. 17: Phototactic movement of Oscillatoria animalis tested at involuted stage (left), after partial growth stage (centre) and after fully grown stage (right).

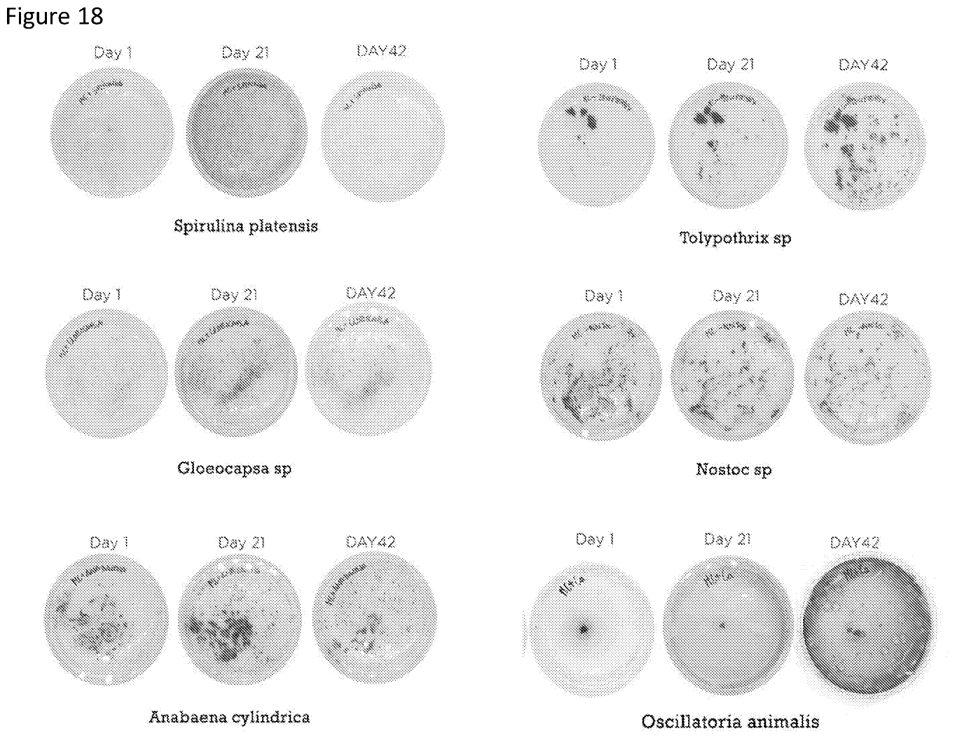

FIG. 18: Growth comparison of different filamentous species of cyanobacteria as mentioned on 6% Methylcellulose gel over a 42 days period.

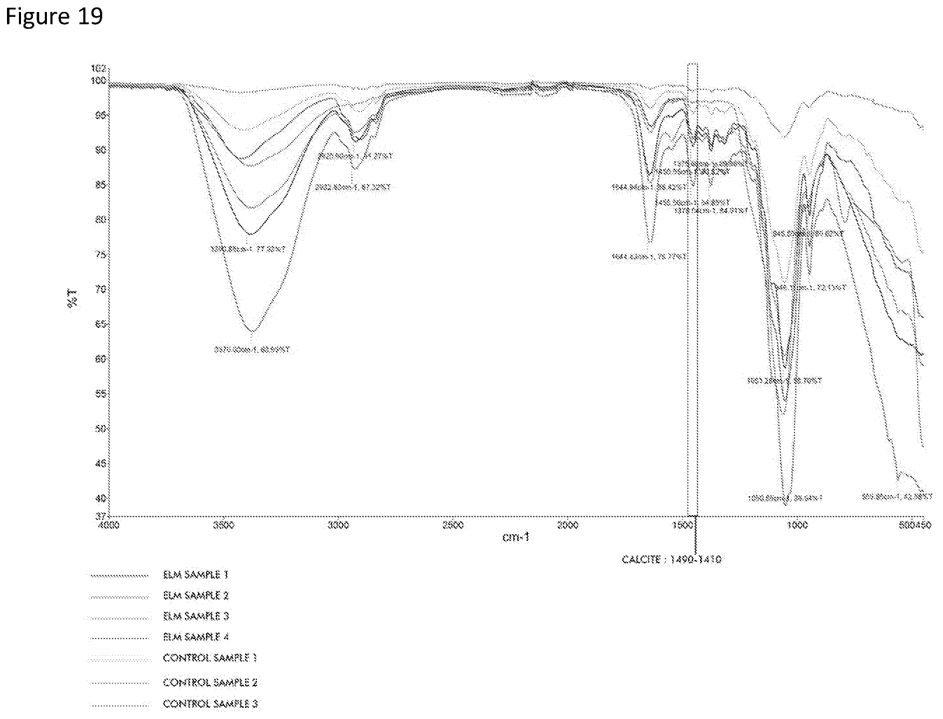

FIG. 19: FTIR graphs comparing the composition of ELM samples and control samples and showing the presence of Calcium carbonate minerals.

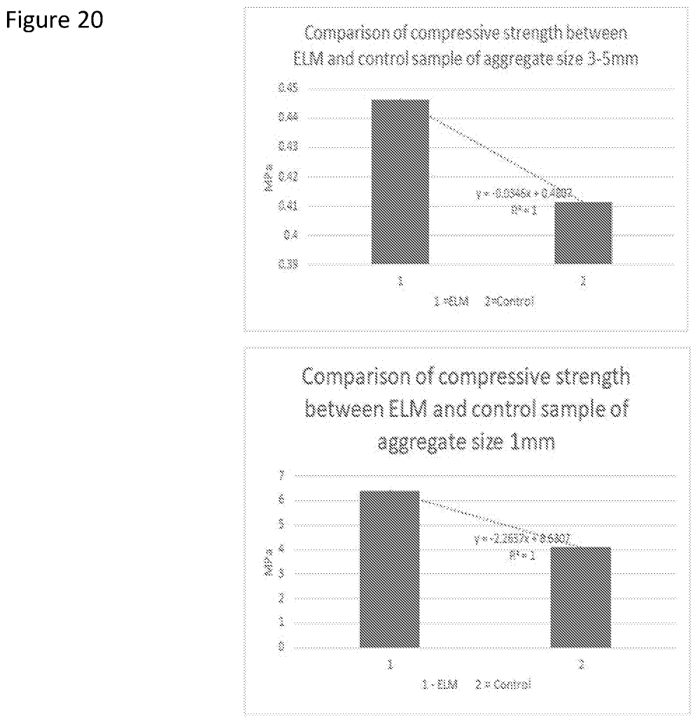

FIG. 20: Graphs showing comparison of compression strength between ELM samples and control samples.

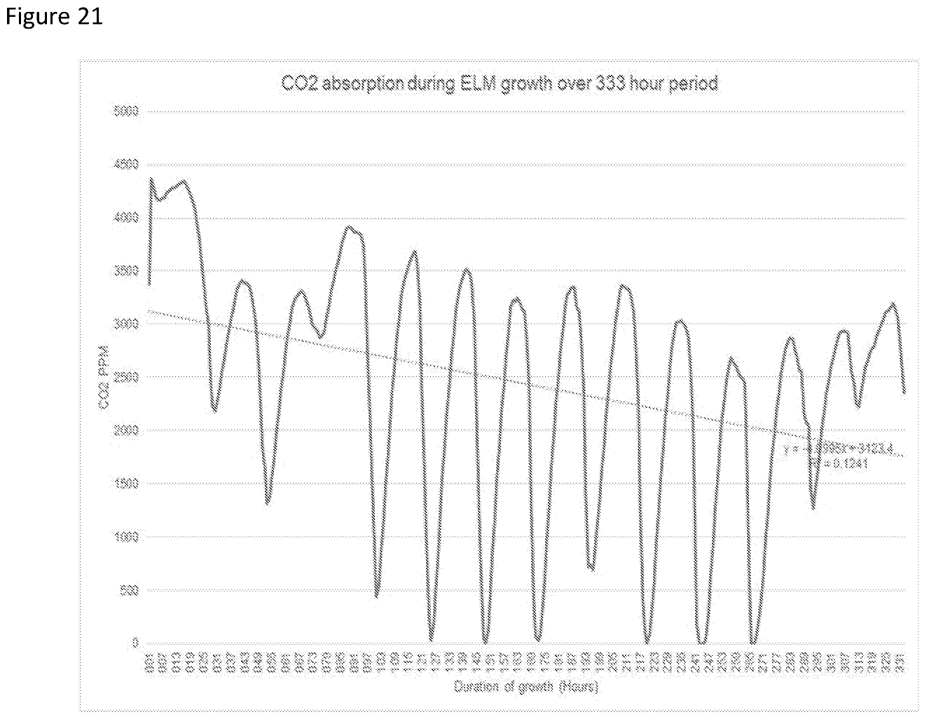

FIG. 21: Graph of CO₂ PPM level over 333 hours of growth period of ELM.

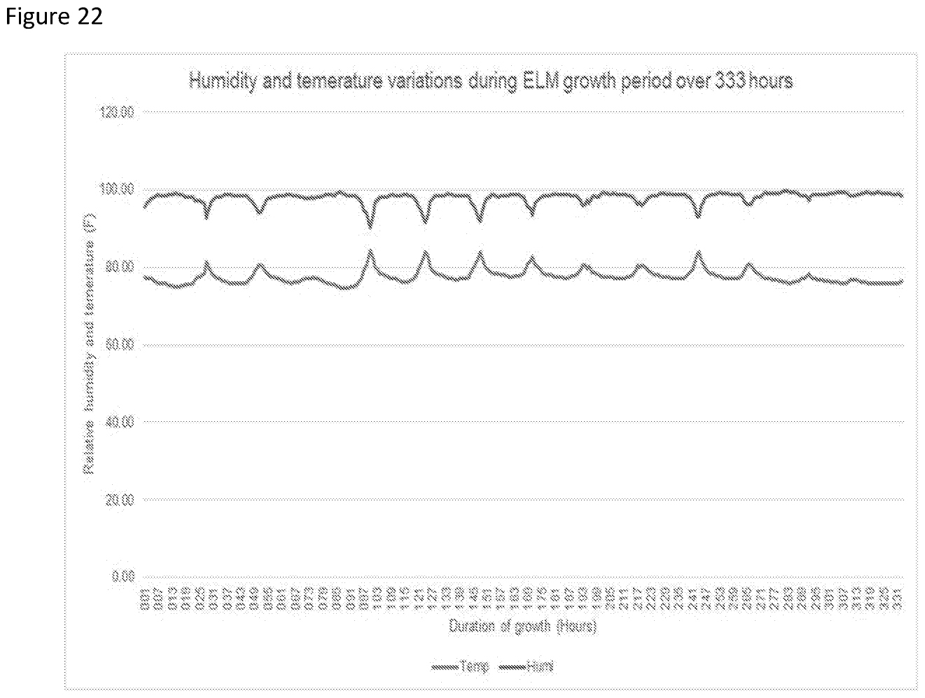

FIG. 22: Graph of Temperature and Humidity patterns inside the casting box over 333 hours of growth period of ELM.

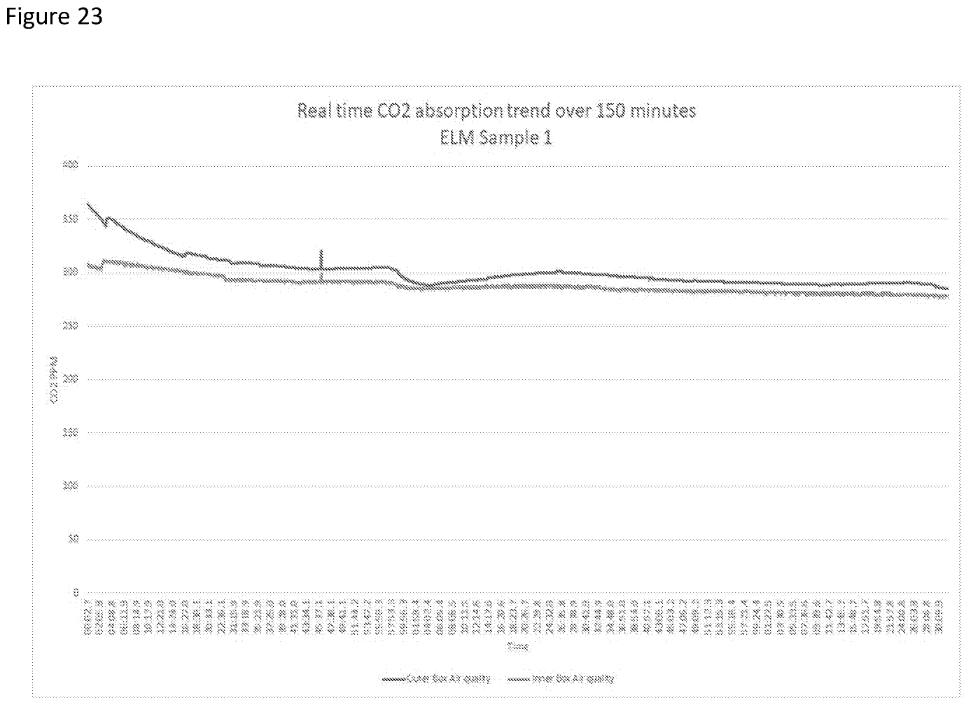

FIG. 23: CO₂ absorption trend of cured ELM sample 1.

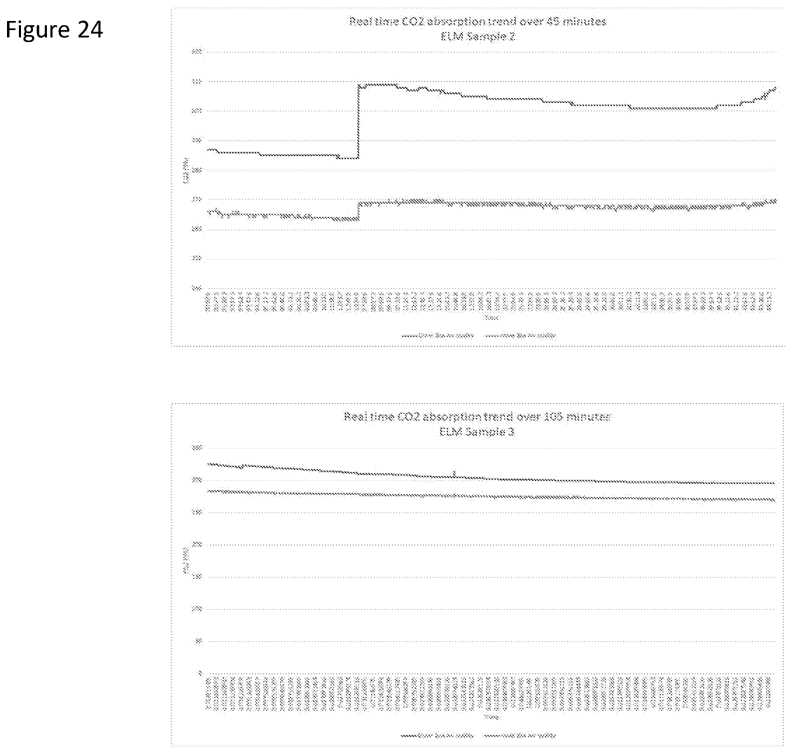

FIG. 24: CO₂ absorption trend of cured ELM sample 2 and sample 3.

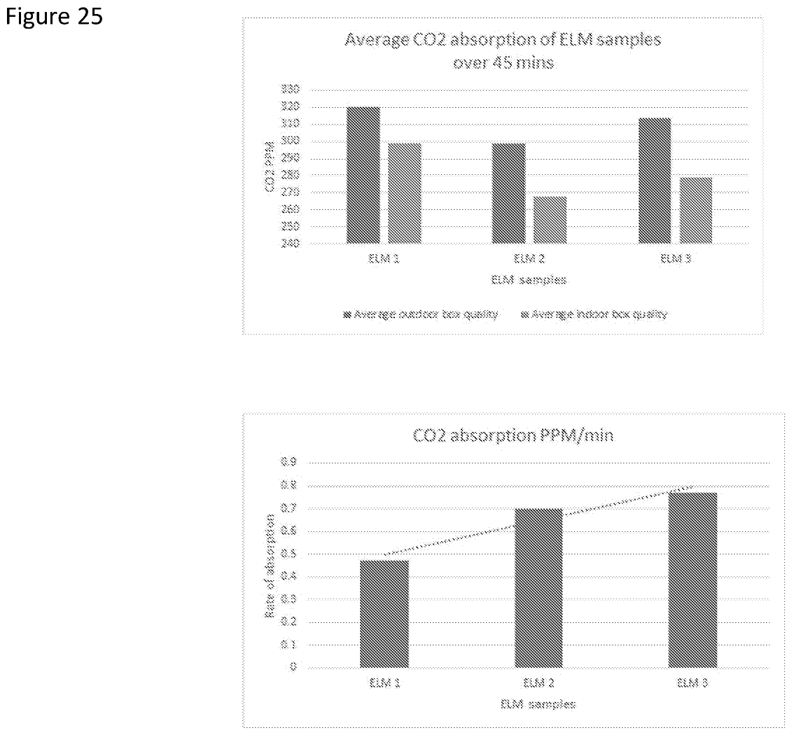

FIG. 25: Comparison of CO₂ absorption trend of the three ELM samples.

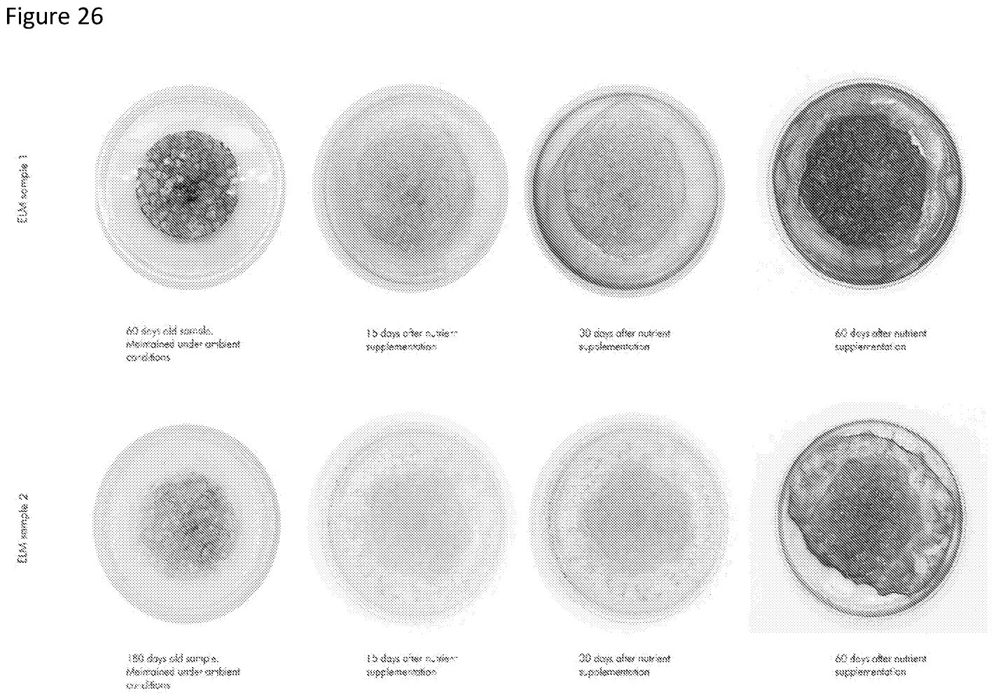

FIG. 26: cellular viability and regenerative capacity test. 50 days old matured PB-ELM prototype kept at natural exposed room temperature conditions supplemented with BG11 nutrient media. New biofilm growth was observed sprawling out of the PB-ELM after 14 days indicating active cellular viability. Oscillatoria animalis biofilm revival observed after 6 months of dehydration. Fully grown agar gel biofilm was dehydrated over a period of 6 months. Visible biofilm patterns vanished however isolated green dormant cells were spotted. After nutrient supplementation of dehydrated gel, new biofilm growth was revived within 2 months.

### EXAMPLE 1

### Bottom-Up Tissue Engineering Approach to Develop Stromatolite-Inspired Scalable Regenerative Photosynthetic Biomineralized Engineered Living Material (PB-ELM).

### 1. Introduction

Contemporary advances in fields like biotechnology, material science and artificial intelligence are opening pathways to new transdisciplinary domains of research that were not possible before. Engineered Living Materials (ELMs) is one such emerging and rapidly developing domains. ELMs are smart biologically augmented materials whose assembly and properties are governed by living cells.[^{1,2}] Historically materials in human civilization has been looked at as pre-existing inanimate elements that can be extracted, reshaped and re-combined through purely physical means such as heating or pressurizing. Though these processes have achieved tremendous feats to produce the incredible assortment of materials that exists today, this approach primarily ends in results that have fixed physical and chemical properties. Moreover, they are often a one-way energy transfer mechanism and therefore unsustainable considering the global environmental challenges that we face today. On the contrary, all living systems operate through a parallel mechanism where biological materials-proteins and tissues, needed for their sustenance are grown or autonomously assembled bottom up. Biological materials are hierarchically graded and therefore extremely efficient. They are responsive to the environmental conditions and therefore inherently flexible in their properties, or self-healing and regenerative. And these materials fit within larger cycles of energy flows that make them environmentally sustainable[³]. The field of Engineered Living Materials is driven by the aim to embed these complex properties that are present only in living systems till now into functional designed materials. In doing so, it is also uncovering novel solutions in fields like biomedicine, advanced electronics, soft robotics, and construction[^{4,5,6}].

Since this area of research has recently emerged and present such diverse possibilities, attempts have been made to categorize the developments, create rational taxonomies, and identify particular areas of challenges to make further progress. Nguyen et al and Srubar, W in their extensive reviews of recent ELM developments have identified two main categories based on the approach taken to develop the ELMs. The first category is engineered proteins and biofilms where secondary metabolites and compounds produced by various microorganisms are harnessed, modified[⁷], or introduced synthetically to achieve desired functional material outputs such as self-assembly, surface pattern structuring or bio-sensing[^{8,9}]. This set of ELMs rely heavily on synthetic biology techniques and operates at a microscopic scale[^{10,11}]. The second approach is more top-down where cells and biofilms are suspended in matrixes or in scaffolds to grow and produce desirable outcomes. This approach relies on tissue engineering techniques and produces ELMs that are artificially generated living composites contain biological and non-biological elements.[^{12,13]}

Srubar. W also states key limitations and challenges that exist across the ELM domains at present. The first challenge relates to scaling up since most of the bottom-up approaches work at micro-scales structuring. In certain fields like micro-electronics and robotics, scale might not be an issue however in other industries such as construction it is of vital importance[^{4,5,6}]. The second challenge is time span of production and growth and is directly related to the first one. Since biological process are slow, these ELMs may not be grown in a commercially viable timeline. The third challenge is maintaining long term viability of the cells/organisms[¹⁴]. ELMs based on chemotrophic systems need to be maintained in the right environmental conditions and continuously fed with appropriate nutrients. For systems that rely on genetically modified organisms, biocontainment is an additional challenge during applications. And although ELMs that operate with wild-type organisms do not that face that challenge, natural living systems often operate within a complex network/consortium of other cells and microorganisms. And achieving a multi-species symbiotically operating consortia-based ELM could greatly enhance their properties or solve some of the previous mentioned limitations[^{16,17}]. Finally, one of the significant challenges lie in the fact that this is an extremely new transdisciplinary field of research and therefore there is a lack of interdisciplinarity-trained human resource as well as tools and methods of evaluating and analysing these materials. New tool sets and techniques need to be developed for the study, production, and application of ELMs.

This research aims to demonstrate a novel photosynthetic biomineralized engineered living material (PB-ELM) that surpasses every existing limitation identified above. This PB-ELM is inspired by natural stromatolites that are biomineralized rock formations made by photosynthetic cyanobacterial communities[¹⁸]. The process of carbonate precipitation in such systems are known to be linked to the release of extracellular carbonic anhydrase enzyme produced as a means of their carbon concentrating mechanism[¹⁹]. However, biofilm formation by filamentous cyanobacteria occur as a 2-dimensional film under natural conditions therefore the process of the stromatolite growth takes hundreds of years to form[²⁰]. Tissue engineering principles were adopted to derive a hydrogel composition that can act as a scaffold for the cyanobacterial biofilm to overcome the 2-dimensional nature and enable to 3-dimension biofilm growth. Naturally abundant, hygroscopic, light transmitting aggregates like SiO₂ were suspended in the hydrogel. A biomineralized photosynthetic living material was thus grown bottom-up in a span of weeks. Custom made computer vision machine learning programs were developed to analyses and study the health of the ELM that can aid in large scale production of the material. The derived data from the analysis programs was digitally scripted to generate algorithmic designs of larger prototypes. Fabrication techniques like additive manufacturing and vacuum forming were used to make molds and successfully test the scalability of the BP-ELM by growing designed meso-scale prototypes. This material is rigid, lightweight, translucent, and actively photosynthesizing and thus presents a revolutionary opportunity to develop carbon negative living architecture to alter the course of climate change.

### 2. Discussion

### 2.1 Cyanobacteria, Stromatolites and Biomineralization:

It is known that the atmospheric conditions in the Precambrian era (4.5 billion years ago) of the Earth was dominated by Carbon Dioxide and hostile to the existence of any life forms. Cyanobacteria are gram-negative eukaryotic bacteria that are known to have been one of the first species to have emerged on the planet in the Archean era with the extraordinary ability to photosynthesize.[²¹] They could use the energy from the sun to convert available environmental elements as nutrients and sustain a living metabolism. This is known to have had an incredibly significant role in the evolution of the present-day biosphere by converting the carbon dioxide rich atmosphere to an oxygen rich environment that supports all other life forms today.[²²] The sequestering of carbon dioxide by cyanobacteria occurs in two primary modes—the first is via Calvin Benson Bassam (CCB) cycle during photosynthesis where CO₂ is captured and converted to organic compounds and the second is via carbonate mineralization where CO₂ is converted to carbonate minerals which is evident through the occurrence of stromatolites[²³].

Stromatolites are biomineralized living rock formations made by cyanobacterial microbial mats. The process of carbonate mineralization in stromatolites is known to be associated to the carbon concentrating mechanism (CCM) of cyanobacteria and the production of extracellular Carbonic Anhydrase enzyme.[^{19,24}] CCM is a ubiquitous strategy evolved by cyanobacteria to use CO₂ dissolved in water. Dissolved CO₂ exists as carbonic acid (H₂CO₃) which disassociates to form bicarbonate ions (HCO₃) which can be assimilated by the cyanobacteria for photosynthesis (Equation 1). The equilibrium of these ions is known to be affected by the pH of the medium with higher pH shifting the equilibrium towards CO₂ and lower pH shifting it towards the HCO₃. The method herein may be at a pH appropriate for producing HCO₃. Carbonic anhydrase catalyses the interconversion of CO₂ to HCO₃ causing a super saturated condition in the microenvironment[^{19,23,24}]. The role of Carbonic anhydrase is significant as it is known to be one of the fastest acting enzymes with 106 conversions per second and it has been found to enable up to 40% more carbon sequestration.[^{24,25}] In the presence of Ca+ ions in the environment, these bicarbonate ions can form Calcium carbonate however there exists significant kinetic barriers for this reaction to occur[²³] (Equation 2, 3). The outer membranes and extra cellular polysaccharide layers of cyanobacteria are known to have a net negative charge. This physical condition overcomes the kinetic barriers by forming a nucleation surface and allows the precipitation of calcium carbonate minerals[²³]. As inorganic inert aggregates particles like sand are trapped in the microbial mats, this mineralization causes a binding phenomenon thus resulting in a lithified rock formation.

CO₂+H₂O⇄HCO₃⁻+H⁺. [Equation 1]

Ca²⁺+2HCO₃⁻⇄Ca(HCO₃)₂, [Equation 2]

Ca(HCO₃)₂⇄CaCO₃+CO₂+H₂O. [Equation 3]

Microbial precipitation of calcium carbonate has been used in geological engineering processes as well as to develop engineered living materials for the building industry[^{26,27}]. However, most of these systems depend on chemotrophic urease-induced pathways[⁶]. Heveran et al demonstrated for the first time (2020) that this chemical process can be adopted to make engineered living building material by suspending sand aggregates in a gelatine gel and mixing it with pre-cultured unicellular Synechococcus cyanobacteria cells[²⁸]. The formation of calcium carbonate minerals was detected through electron microscopy and spectroscopy and it was revealed that the fracture strength of these biomineralized ELM samples were higher than abiotic samples. This was a top-down approach and it validated the applicability of the process; however, it does not possess the advantages of the bottom-up stromatolite process that exists in nature. Stromatolites are made primarily by filamentous mat-forming cyanobacterial species[¹⁸]. Most of these species have the ability of gliding motility and are thus able to move through the stromatolite to regulate their need for light and nutrient[²⁹]. This allows the cyanobacteria to inhabit the cyanobacteria for extremely long duration (hundreds of years) and endows the stromatolite with the properties of regeneration and active physical growth[²⁰]. In the approach adopted by Heveran et al, it was possible to maintain the viability of the ELM for a maximum of 30 days by incubating the samples under simulated heat and humidity conditions which allowed for successive regeneration of the material within the maintained period. But natural long-term viability of the cyanobacteria could not be achieved due to physical immobilization of the cells and inherently unfavourable light conditions created by the opaque aggregates. The simulated conditions and requirement of separate cell culturing presents pragmatic limitations in terms of industrial applications and production. The objective of this research was to harness the same mineralization process but through a bottom-up structuring strategy instead that would achieve identical stromatolite properties.

### 2.2 Species Selection and Study Methods

Since the objective was to follow a bottom-up approach, the first step was to identify an appropriate species of cyanobacteria. Six different available species of filamentous cyanobacteria species were tested-Anabaena cylindrica, Nostoc sp, Tolypothrix sp, Gloeocapsa sp, Oscillatoria animalis and Spirulina platensis. Each of these species are known to have different morphological and physiological characteristics like false branching, heterocyst formation, EPS production and gliding motility etc. The species were cultured under three different conditions-standard agar BG11 media, agar BG11 media with 0.1M calcium chloride concentration and BG11 liquid media with sand aggregates. Finally, Oscillatoria animalis was chosen as the model species due to its fast growth rate and complex interwoven biofilm pattern emerging because of its gliding motility behaviour. The complex interwoven biofilm patterns exhibited an inherent tendency to bind sand particles.

Oscillatoria is a filamentous cyanobacteria species that is named after its oscillation in its movement. It is commonly found near freshwater troughs, but it is also been reported under terrestrial conditions. It is known to produce long trichome filaments and reproduces through fragmentation[³⁰]. The biofilm patterns are understood to be an emergent phenomenon of its helical fibril layer on its trichome surfaces that create a circular force when gliding over surfaces. However, it is not a commonly studied species and not much is known with regards to its complex growth behaviours. So, its growth characteristics were observed under various biochemical and biophysical conditions such as its interaction with different physical scaffolds in liquid media. It was understood that due to its 2-dimensional film forming nature, conventional growth evaluation techniques such as optical density measure were not possible. At the same time, since the broader aim was to evaluate the growth under applied outdoor conditions of the ELM, a need for custom-designed evaluation tool was identified with regards to the primary challenges in the ELM domain as mentioned previously.

Two computer vision image analysis tools were developed as a non-destructive means of evaluating Oscillatoria animalis biofilms. The first tool evaluated the growth rate and extent of the biofilm by calculating the total green pixels and excess green index of the culture images. Excess Green Index (ExG) typically used to measure plant biomass against soil and residue for remote sensing in ecological studies[³¹]. The function is defined to normalise the red, green, and blue intensities (sometimes called the chromatic coordinates) of the image and calculate the ExG index for each pixel in a given image. Both the Total green pixel and the ExG parameter graphs co-related to the experimental observations however by testing images from different cultures it was identified that the ExG index parameter was more accurate. The objective in this case was to evaluate the growth a biofilm/colony level and therefore this tool cannot predict the biomass growth of the culture but the quantitative data can help understand the growth rate of the culture and identify critical growth levels.

The second tool evaluated the photosynthetic health of the biofilm. The Carbon concentrating mechanism and the production of carbonic anhydrase is known to closely linked to the photosynthetic activity of the cyanobacteria[³²]. Inhibition of carbonic anhydrase enzyme has been reported to decrease both net and gross photosynthetic productivities[²⁴]. Photosynthetic activity is also associated with the light harvesting physcobilisome pigment complex that give cyanobacteria their characteristic blue-green color[^{33,34}]. It was noticed in the initial experiments that certain cultures under nutrient deficient or adverse light conditions changed colors indicating a deterioration in their photosynthetic health. This change is color is subject to human eye, but definite values could be derived through digital analysis of Hue, Value and Saturation (HSV) values of the images[³⁵]. Both the RGB and HSV values of these dominant colors were extracted[³⁶]. It could be noted that under nutrient sufficient conditions, the hue values ranged from 60-80 while under nutrient deficient conditions the hue values were above 80. This indicated a threshold color value for optimum photosynthetic activity and thus carbonic anhydrase production.

### 2.3 Hypothesis and Primary Developments

Dupraz et al have classified biomineralization processes into three different categories. The first is biologically controlled mineralization which occurs in bones and shells, the second is biologically induced mineralization such as in stromatolites and the third is biologically influenced mineralization where the mineralization is a geo-physical phenomenon, but the presence of organic matter influences the crystal morphology and composition[¹⁹]. Although the processes vary, biominerals formed in each of these conditions are known to fit in the criteria of true minerals biogenic calcium carbonate possess different characteristics as opposed to their inorganic counterparts. Biominerals have unique crystal morphology depending on the matrix and the structurally they are agglomerations of crystal infused with organic matter. They are also known to demonstrate high nanoindentation hardness compared to abiotic calcium carbonate[³⁷]. In the field of artificial tissue engineering, hydrogels, which are 3 dimensional networks of hydrophilic polymers, have been tested to grow mineralised tissues with results akin to natural tissue such as bones[^{38,39}]. Nindiysari et al and Asenath-Smith et al have shown that minerals formed in hydrogel matrixes have similar biomimetic morphology and hybrid organic-inorganic compositions[^{40,41,42}].

An alternative process of stromatolite formation was hypothesised considering an artificial microbial tissue engineering approach. One of the most significant hurdles was that cyanobacterial biofilms always grow in 2-dimensional sheets under natural conditions, so the stromatolites form one microlayer at a time and thus takes hundreds of years to attain significant depth. Alternatively, all the aggregate layers could be taken at once and suspended in a hydrogel matrix with nutrients and calcium ions and inoculated with the cyanobacteria. If the cyanobacteria could grow through the hydrogel matrix and surround the aggregates, the release of extracellular carbonic anhydrase will enable the precipitation of calcium carbonate crystals within the aggregates and thus achieve a biomineralized living material in a significantly short span of time. Hydrogels are generally used to immobilize cells and so a range of different hydrogel compounds were tested to observe the interaction-Agar, Gelatine, Chitosan, Sodium alginate, Methylcellulose, Carrageenan, Silk fibroin and Sodium Polyacrylate (FIG. 1). A significant breakthrough was achieved by discovering that Oscillatoria animalis and methylcellulose had a unique relationship and the cyanobacteria biofilm could grow in a three-dimensional structure of the matrix (FIG. 3). However, methylcellulose gel is known to increase in viscosity at lower temperatures. Partial biofilm growth was observed in crosslinked sodium alginate[^{43,44}]. In all other hydrogel compounds, there was only surface growth of the biofilm. An optimized hydrogel composition ratio of methylcellulose-sodium alginate (6:1) was arrived at after subsequent testing which allowed fast 3-dimensional growth at the same time had a low viscous consistent gel structure. This composition is thermally stable as sodium alginate is known to have high gel viscosity at lower temperatures contrary to methylcellulose[⁴⁵]. The reason for this unique interaction could not be ascertained at this point. One of the possible explanations could be that methylcellulose-sodium alginate hydrogel is known to be thixotropic, it reacts like a solid to perpendicular forces but like a liquid to planar forces[⁴⁶]. Since Oscillatoria is known to have a characteristic oscillating tendency, the vibration makes the hydrogel behave like a liquid in its microenvironment which makes it possible to grow within in a 3-dimensional form but as a whole it behaves like a semi-solid gel.

The next step was to test the interaction of the Oscillatoria animalis biofilm with different kind of aggregates-cellulose based aggregates like cotton fibres, Luffa actutangula, sand, activated carbon and amorphous silica (SiO₂). It was noticed that aggregates with a high micro-porous surface capacity such as activated carbon and SiO₂ exhibited inherent binding tendency with the biofilm. Microscopic imaging revealed that the cyanobacteria trichomes could interweave through the porosity of adjacent particles resulting in a continuous mat (FIG. 2). Activated carbon has a micro-surface capacity of 3000 m²/g however it is dark opaque and not ideal for light transmission. SiO₂ has a lower micro surface capacity of 800 m²/g but it is translucent when dry and transparent when wet. Silica is also naturally hydroscopic and widely used as a desiccant because of its ability to adsorb moisture from air[^{47,48}]. Ciriminna. R et al have previously suggested that these inherent properties of amorphous silica are ideal to act as a base for developing advanced materials[⁴⁹]. Also, compared to river sand which is becoming a scare resource for construction, silicon dioxide is known as the second most abundant mineral on Earth and an great untapped resource potential. Thus, Silica was chosen as the ideal aggregate for the ELM.

Combining the observations from these two developments, SiO₂ crystals were suspended in a methylcellulose-sodium alginate gel with BG11 media and 0.1M calcium chloride solution. A volume of 2.94 m³ of the material was grown over a period of 12 days under room temperature and dehydrated over 4 days (FIG. 4). A rigid, light weight, translucent, hydroscopic prototype of an actively photosynthesizing biomineralized engineered living material was achieved (FIG. 5). This approach is bottom-up as the cells were not separately cultured and mixed into an arbitrarily chosen aggregate matrix. The growth of 3-dimensional biofilm is organic and micro-structural interaction and biomineralization of the aggregates is controlled by the cyanobacteria. Microscopic images revealed active cyanobacterial biofilm forming binding bridges between the aggregates (FIG. 6).

### 2.4 Scaling Up and Meso-Scale Prototypes

The growth of the ELM samples is influenced by two factors, the amount of inoculum used and the volume of the sample. Using the previously mentioned computer vision image analysis programs, regression analysis-based machine learning model was derived to predict the growth and behaviour of larger prototypes. Because of the firm forming tendency of Oscillatoria animalis, it is difficult to quantify the exact amount of inoculum added to grow the ELM in terms of cellular biomass. But the amount of surface area of the ELM inoculated can be quantified by analysing the ExG index (FIG. 7, 8). By plotting the dependent variable of time against the independent variable of volume and ExG index, a multiple regression analysis graph can be plotted and the relationships between the factors were identified (FIG. 9). With data from 9 samples it was identified that R (ExG)=−0.007 and R(Volume)=0.210. This value can be made more accurate by analysing more samples and an accurate estimation of how much inoculum ExG will be needed to grow a given volume of the ELM in a desired timeline can be derived.

At the same time, HSV colour analysis of the BP-ELM samples was conducted to understand the photosynthetic health of the ELM (FIG. 10). It was observed that the pigment expression of the cyanobacteria was dependent on nutrient availability and the amount and duration of light exposure. This data can be correlated with material strength analysis data of the samples and a similar regression analysis can be done to derive the relationship between the photosynthetic pigment expression and the material strength. This could help in visually identifying the mineralization extent in larger prototypes and add nutrient supplements when needed. At the same time, environmental simulation software can be used to analyse the light and temperature conditions in which the ELM will be installed, and an ideal morphological expression of the ELM can be designed for desired growth and mineralization.

This was tested in a software called Houdini-SideFx. A mathematical modulo operator-based algorithm was first used to generate a design expression. The surface exposure to light of the design was identified by analysing the curvature of the surfaces under outdoor conditions. The HSV colour data was then added to the script to produce a visualization of the grown BP-ELM (FIG. 11). Various iterations were created. The designs were then 3-D printed and vacuum forming was used to make transparent moulds of size 16×8×2 cm (FIG. 12). A maximum mould depth of 3.5 cm was tested (FIG. 13). These moulds were then incubated with BP-ELM and allowed to grow for a projected period of 35 days under room temperature and light conditions-average temp=15 degrees and average light intensity=2400 lux. Digitally designed BP-ELM samples were thus successfully grown and derived (FIG. 14).

### 2.5 BP-ELM Properties

Physical properties—The BP-ELM is rigid, hard and light weight, approximately 3 g/cm³. The aggregate types, type such as-amorphous and crystalline (Quartz) SiO₂ and their ratio can be changed to derive version of different physical properties (FIG. 15). This makes the BP-ELM an ideal cladding material and given the ability to be casted in desired digitally generated forms, it can thus be used as a photosynthetic architectural skin. Although the formation of minerals and the enhancement of fracture strength achieved by photosynthetic biomineralization was confirmed by Heveran et al, this BP-ELM is yet to be scanned with electron microscopy and analysed with Dynamic Material Analyser. Once the exact compressive strength, fracture strength and bending stress is identified, ideal application scenarios can be designed with appropriate reinforcements for more load bearing architectural structures.

Cell viability—This BP-ELM is expected to surpass the viability of the preceding ELMs and remain active for long-durations under natural conditions for a variety of reasons. Oscillatoria animalis has been reported as a resilient species found both in water throughs as well as under terrestrial conditions unlike unicellular species. The primary aggregate, non-crystalline SiO₂ is naturally hygroscopic and transparent. It turns transparent when completely moist. This inherent property creates ideal conditions of micro-humidity and light transmission for the cyanobacteria to sustain its own metabolism. At the same time many filamentous species of cyanobacteria species are known to be able to fix nitrogen from air and form dormant cells that can survive extremely long durations of harsh conditions[⁵⁰]. To test the viability, a 50-day old BP-ELM sample, kept under room temperature conditions and without any nutrient supplements was immersed in liquid BG11 media. It was noticed that cyanobacterial biofilms were sprawling out of the material within a span of 14 days (FIG. 37). This confirmed that the material is self-healing and regenerative under natural conditions. This was significantly higher viability period and under natural non-simulated conditions than reported by Heveran et al with Synechococcus, Achal et al with Bacillus megaterium and Bundur et al with Sporosarcina pasteurii. At the same time, to test the dormant cell formation by Oscillatoria animalis, the cyanobacteria were grown on a standard BG11 5 mm diameter agar petri dish. The biofilm was grown until it saturated the surface. Microscopic images revealed the formation of darker, cells with thicker cell walls which are typical identifying characteristics of cyanobacterial dormant cells (spore-like cells)[^{51,52}]. The agar plate was then allowed to completely dehydrate for 6 months. It was noticed that after 2 months, the biofilm disappeared as the cells died but the isolated circular dormant cells remained green (FIG. 16). The dehydrated gel membrane was then immersed in nutrient BG11 media after 6 months, and it could be seen that whole surface turned green after 1 month of nutrient supplementation indicating the regeneration of the biofilm form the dormant cells (FIG. 37).

Optical property—In addition to these properties, the moisture reversible translucency of the BP-ELMs acts as an additional optical property which has not been present in other building living material (FIG. 15). This property adds an additional dimension that can be designed for architectural applications.

Phototactic bio-programming—One of the most prominent features of ELMs is that they are biologically programmable. Most current ELM systems rely on synthetic genetic engineering methods to program the properties. However, that creates restriction sin terms of biocontainment and natural applications. In this approach we suggest a natural method of programming the controlling the external stimuli of the cyanobacteria to direct the growth of the biofilm. Cyanobacteria is known to exhibit phototactic behaviour and respond to light direction, intensity, and wavelength. Therefore, designed light exposure of the BP-ELM can be used to control the growth direction of the Oscillatoria animalis biofilm and thus achieve programmed biomineralization. This feasibility of this hypothesis was tested by selectively exposing Oscillatoria biofilm cultures on agar petri dishes. To test the phototactic behaviour, one half of the petri dish was covered with black tape and three conditions were tests-inoculated stage, partially grown stage and fully grown stage. In the inoculated stage, it was observed that the biofilm would only grow towards the area that was exposed to light and there was almost a noticeable line of separation between the areas. In the partially grown stage, it was noticed that the part of the biofilm under the covered are slightly diminished and there was significantly higher growth towards the exposed area. In the fully grown stage, there was a clear distinguishable line of variation in density of the biofilm between the covered and exposed areas (FIG. 17). These changes were noticed within a period of 14 days. Similar test was later done by simply keeping the cultures away from direct sunlight but facing a unidirectional window light source. The phototactic response was noted in transition phases between liquid to solid media as well as solid to liquid. This confirms the possibility of bio-programming the BP-ELM with phototactic manipulation.

Organism consortia—Finally, one of the challenges in the domain of ELM is achieving a symbiotic multi-species interaction. Natural stromatolites although mainly formed by cyanobacteria, are known to be composed of complex microbial mats with many autotrophic and chemotrophic organism functioning as a community. These interactions have been sufficiently recorded and can be tested on the BP-ELM. However, as a means of testing the possibility of a consortia, five other filamentous species of cyanobacteria were tested under the same ELM conditions—Anabaena cylindrica, Nostoc sp, Tolypothrix sp, Gloeocapsa sp and Spirulina platensis. It was noticed that Spirulina exhibited significant growth and Anabaena exhibited partial growth in a short period in the same BP-ELM prototype conditions. However, their growth was not sustained after more than 21 days. Tolypothrix exhibited slow and steady growth but it has a natural tendency to grow as clumps and not as a film (FIG. 18). These species therefore represent the possibility of designing an organism consortium with variable life spans. Some species that that grow fast, while others grow slow and some can form films, dormant cells and heterocysts for long term survival of the BP-ELM.

### 3. Experiments and Assessment Procedures

### Preliminary Study Experiments

Oscillatoria animalis and all the other cyanobacterial species were obtained from Sciento culture lab. Subsequent liquid cultures were made for each species using 35 ml BG11 media (supplemented with BG11 trace elements) in falcon tubes. These cultures were grown at ambient room temperature of 15 degrees against a sunlight of 2400 lux. For growth rate study experiments, a set of 1.5% agar plates were created with BG11 media and with 0.1 M calcium chloride dihydrate as control and test respectively. Each place was inoculated with 1 ml of the different cyanobacteria species cultures. Observations were recorded every 72 hours through digital and microscopic imagery. For the sand binding test, 1.8 mm dia (avg) river sand was procured and sterilized in an autoclave. 8 g of the sand was poured into a 9 mm dia petri dish such as to create a thick surface layer of sand. The plates were supplemented with 4 ml of BG11 media until the sand layer was visibly moist and each plate was inoculated with 1 ml of each cyanobacteria culture. Observations were recorded every 72 hours though digital and microscopic imagery.

### Hydrogel Experiment

Nine different Hydrogel compounds were chosen-Agar, Gelatine, Chitosan, Sodium alginate, Methylcellulose, Carrageenan, Silk fibroin, Laponite and Sodium Polyacrylate. 100 ml of each gel was made in separate beakers with BG11 media. Since each of these compounds have different gelling conditions, respective amounts of the compounds were weighed and added under conditions as mentioned in Table 2. 0.1 M CaCl₂·2H₂O solution was added to each gel and triplicates of each gel was inoculated with Oscillatoria in 5 mm dia petri dishes. One control petri dish without the addition of calcium chloride dihydrate was inoculated for each gelling compound. To inoculate approximately same amount of cell, the biofilm in the falcon tube cultures were disintegrated by manual shaking for 5 mins and 1 ml of culture was added to epindorph tubes and centrifuged under 3000 rpm for 15 mins. The inoculated petri dishes were grown under the same ambient room temperature and light intensity. Observations were recorded after every 48 hours for 10 days by digital imagery. A second set of the experiment was conducted to grow the gels under exposed non-sterile conditions by removing the petri dish cover to study the contamination tendency of each gel since the final objective is to obtain a gel that can be used in outdoor applications.

3-dimensional biofilm growth was observed only in the 6% Methylcellulose gel and partial growth was observed in 3% cross-linked sodium alginate (not in the control sodium alginate gel without calcium chloride). A follow up experiment was conducted to optimize the gelling compound by adding 1%, 2% and 3% sodium alginate to the 6% methylcellulose gel with 0.1 M CaCl₂·2H₂O and inoculated in the same manner as mentioned above. It was observed that 3-dimensional biofilm growth was demonstrated with 6% Methylcellulose and 1% Sodium alginate cross linked. This gel mix was thus chosen as it enabled best gel consistency with optimum growth.

### Aggregate Experiment

Six different aggregates were tested based on initial observations. Early study experiments demonstrated that Oscillatoria exhibited binding tendency with 2 mm dia sand particles, therefore porous aggregates like activated carbon (5 mm dia) and silica (SiO₂) (5 mm dia) were chosen. From the hydrogel experiments it was observed that Oscillatoria animalis demonstrated affinity for Methylcellulose and therefore 3 cellulose based aggregates were chosen-cellulose sponge, Luffa acutangular scrub and cotton wicks. Each of these aggregates were sterilized and adjusted to fit the volume of 5 mm dia petri dish. The aggregates were supplemented with 5-10 ml BG11 media until visibly wet and inoculated with Oscillatoria animalis cells after centrifuging as mentioned above. Observations were recorded after every 48 hours though digital imagery as well as microscopic imagery. Kern optical microscope was used for the purpose. Biofilm growth was observed in all three porous aggregates of sand, activated carbon and silica and n growth was observed with the cellulose based aggregates. While the best binding tendency was exhibited with activated carbon due to its high micro porosity, silica was chosen as the ideal aggregate as its additional properties of hygroscopy and translucency provided ideal conditions for long term growth.

### Prototype and Cellular Viability Experiment

Based on the observation results from the hydrogel and aggregate experiments, a prototype experiment was set up. 8 g of 5 mmm dia SiO₂ aggregates were added to 12 ml3 of 6:1 Methylcellulose and Sodium Alginate gel mix with 0.1M CaCl₂·2H₂O and inoculated with Oscillatoria animalis cells with 1 ml of centrifuge isolated cells. Triplicates samples were incubated under ambient room temperature and light conditions. After 12 days, the biofilm was seen to have grown completely through the volume. The cover lids were removed, and the samples were allowed to dry under exposed conditions at room temperature. After 4 days of drying, the samples were completely dehydrated and were demoulded from the petri dishes. The BP-ELM prototypes were obtained.

The prototypes were kept in exposed condition under room temperature without any addition of nutrient media for 50 days. The samples were observed to maintain their green. The sample was the taken in a larger petri dish of 9 mmm dia and 5 ml of nutrient media was added to immerge it. After 14 days, biofilm growth was observed to cover the surface of the 9 mm petri dish emerging from the prototype sample indicating the viability of the cyanobacteria withing the material.

### Phototaxis Experiment

To test the phototactic behaviour, 15% agar gel with BG11 media supplemented with 0.1M calcium chloride dihydrate was used. The objective was to test whether it is only growth that responded to light or a grown biofilm could respond to light as well. Three sets of plates were inoculated after 14- and 7-days interval between them. On the 21^{st} day, the first set of plated contained completely grown Oscillatoria biofilm, the second set has partially grown biofilm and the third set had just inoculum. One half of the plates were covered with black tape across the diameter. The exposed parts of the plates were left facing a unidirectional window light. After 21 more days of growth, the tape covering was removed to evaluate the growth.

### Organism Consortia Experiment

Identical conditions of the BP-ELM grown with Oscillatoria was used to test the growth of five other species-Anabaena cylindrica, Nostoc sp, Tolypothrix sp, Gloeocapsa sp and Spirulina platensis. 6:1 Methylcellulose-Sodium alginate gel prepared with BG11 media and supplemented with 0.1 M CaCl₂·2H₂O was used to inoculate triplicates of each species. Growth was recorded every 72 hours for 30 days. Spirulina and Anaebena demonstrated growth initially but happened to deteriorate after 21 days. Tolypothrix exhibited very slow and steady growth for more than 30 days.

### Image Analysis

Set-up and equipment—To keep several aspects of the image data such as resolution, brightness and contrast consistent over the period, a POLAMD mini portable photography tent was used as the image collection space. Samsung M30 was used as the primary digital camera. Microscopic images were collected in a Kern microscope and later from JUISON portable microscope. Python was largely used as the programming language to create the image analysis programs with libraries like Skimage, Matplotlib, OpenCV, Pandas and Skipy to access various functions. Spyder and Jupyter notebooks IDEs from Anaconda distribution were mainly used for scripting.

Growth analysis—Since the Cyanobacteria is vibrant green in color and digital images store image data in 3 dimensional arrays, pixel data of a growing culture can be analysed to approximate the growth. For this purpose, two measurements were done, total green area and Excess green index.

Excess green index is usually used to measure plant biomass against soil and residue for remote sensing in ecological studies was adapted. The function is defined to normalise the red, green and blue intensities (sometimes called the chromatic coordinates) of the image and calculate the ExG index for each pixel in a given image according to the formula given below.

The normalised RGB values (or chromatic coordinates) are given by

$$r = \frac{R}{R + G + B} , g = \frac{G}{R + G + B} and b = \frac{B}{R + G + B} ,$$

where R, G and B are the RGB values given as floating-point representations i.e. pixel values between 0 and 1.

The excess green index is then defined as

$$ExG = 2 g - r - b .$$

A chronological set of images labeled in order were read into the script. The images were split into its red, green, and blue intensity channels and the total green area was calculated. The ExG Index was defined as a function and operated on the images simultaneously. The results were plotted on the graph. A histogram was plotted for the ExG index data and a threshold value was selected from it to visually display the biofilm area and to define the total green area.

ExG function accurately identified the biofilm areas. Both the total ExG index and the Green pixel area graphs co-relate to the chronological order of the cultures i.e increasing value with growth time. Specific quantities of total number of pixels for each parameter was obtained.

Photosynthetic health analysis-Colors in the digital screen are produced in the RGB model which splits the image into three primary Red, Green and Blue intensities and all resultant colors are produced as the addition of the primary ones. Each color is therefore given a set of 3 values of red, green and blue. Though this model is ideal to digitally reproduce life like colors, humans perceive colors in terms of hue, saturation, and value. Digital images can be converted to this HSV model which is commonly represented in a cylindrical format. The hues are represented around the circumference with each value ranging from 0-360. Saturation is represented radially from 0-1 and value is represented along the axis from 0-1 as well. The HSV model when represented in the screen produces visually different colors due to their conversion but it returns a single value for the hue. If images are taken under the same lighting conditions, then value can be considered as a constant and saturation would then reflect density. The HSV representation was therefore selected as the ideal model for the analysis of change in color of the cultures.

Images from cultures that exhibited a change in color over time were selected as subject data to test this computational tool. A set of chronological images were read into the script and converted to HSV color space. All the pixels were then arranged in a 3-dimensional plot according to their hue, saturation and value to identify the major differences between the images (FIGS. 28 & 31). The cells under nutrient deficient condition had a larger range in hue values than the standard condition. Separate histograms were then plotted for each data set of hue, saturation and value. Finally, the method of K-means clustering was used to group pixels under five most dominant colors and were plotted in a color bar with their respective occurrence frequency in the images. Both the RGB and HSV values of these dominant colors were extracted.

TABLE 2
Hydrogel preparation procedures and respective observation of biofilm growth.

| | Hydrogel | | |
|---|---|---|---|
| | compound | Preparation procedure | Biofilm growth observation |
| 1 | Agar | 1.5 g agar was mixed 100 ml BG11 | Substantial surface growth of |
| | | media and heated to 90 degree | biofilm was observed after 21 |
| | | centigrade and 0.1M CaCl₂•2H₂O was | days. The growth on control agar |
| | | added to it. The heated gel was | without calcium was faster than |
| | | poured into plates. Once cooled, it the | with calcium supplemented gel. |
| | | gel was inoculated. | |
| 2 | Sodium | 3 g of sodium alginate was mixed with | No growth was observed in |
| | alginate | 100 ml BG11 media. 0.1M CaCl₂•2H₂O | control. Partial growth was |
| | | was added to it. The gel cross linked | observed in crosslinked calcium |
| | | and thickened. It was spread onto | supplemented gel. |
| | | plates and inoculated. | |
| 3 | Gelatine | 100 ml BG11 media was heated to 80 | No growth was observed. The gel |
| | | degrees and 5 g gelatine was mixed. | turned into liquid under room |
| | | 0.1M CaCl₂•2H₂O was added to it. The | temperature in 21 days. |
| | | hot gel was poured into plates and | |
| | | allowed to cool and inoculated. | |
| 4 | Methylcellulose | 100 ml BG11 media was heated to 80 | Significant 3-dimensional biofilm |
| | | degrees and 6 g methylcellulose was | growth was observed all through |
| | | mixed. 0.1M CaCl₂•2H₂O was added to | the volume. |
| | | it. The hot gel was poured into plates | |
| | | and allowed to cool and inoculated. | |
| 5 | Chitosan | 1% acetic acid was added to 100 ml | No growth was observed. |
| | | BG11 media. 3 g chitosan was added to | |
| | | the acidic media and allowed to stir in | |
| | | a magnetic stirrer at 50 degrees for 1 | |
| | | hr. 0.1M CaCl₂•2H₂O was added to it. | |
| | | 1 ml bromothymol blue was added to | |
| | | the dissolved liquid as a pH indicator. | |
| | | The mixed liquid was poured into | |
| | | plates and 0.1M NaOH was slowly | |
| | | added until the chitosan turned blue | |
| | | indicating a neutral pH and gelled. | |
| 6 | Silk fibroin | 100 ml BG11 media was heated to 80 | No growth was observed. The was |
| | | degrees and 15 g silk fibroin was | susceptible to contamination. |
| | | mixed. 0.1M CaCl₂•2H₂O was added to | |
| | | it. The gel was poured into plates and | |
| | | allowed to cool and later inoculated. | |
| 7 | Carrageenan | 4 g of carrageenan was mixed with 100 | No growth was observed. |
| | | ml BG11 media. 0.1M CaCl₂•2H₂O was | |
| | | added to it. The gel cross linked and | |
| | | thickened. It was spread onto plates | |
| | | and inoculated. | |
| 8 | Sodium | 3 g of sodium polyacrylate was mixed | No growth was observed. |
| | polyacrylate | with 100 ml BG11 media. 0.1M | |
| | | CaCl₂•2H₂O was added to it. The gel | |
| | | cross linked and thickened. It was | |
| | | spread onto plates and inoculated. | |

### 4. Conclusion

In this work we demonstrated a truly bottom-up method to create a scalable, regenerative, photosynthetically active, biomineralized engineered living material that surpasses every limitation currently present in the domain of ELMs. Decisions at each stage from species selection, to matrix configuration to aggregate composition was determined by results from first-hand experimental observations. The chemical process of biomineralization is well known from studies of stromatolites and have been previously tested to make ELM but had never been adapted through a tissue engineering approach as demonstrated. The material is composed of living cells, organic derivative hydrogel compounds and naturally abundant aggregates which make the PB-ELM environmentally sustainable. The fast growth rate of the cyanobacteria enables the production of the material within an industrially feasible time span. The material can be incubated and grown as an integrated system and does not require the separate large-scale cell culturing bioreactors or mixing facilities. The inherent properties of light transmission and hygroscopy of the material as well as the ability of the cyanobacteria to survive under harsh conditions ensures the long-term viability of the material under natural conditions. The custom-made computer vision analysis and machine learning tools developed help in the design application of the material.

The building industry today is known to be one of the largest contributors to global greenhouse gases. And as the world population is set to rise to 9.8 billion by 2050, there is an expected demand for 2 billion more buildings[⁵³]. There is therefore an urgent need to simultaneously find new ways to address these needs and mitigate climate change. This PB-ELM presents a revolutionary opportunity to design carbon negative living architecture that can alter the course of climate change. At the same time, since cyanobacteria are known to be the primary organisms to have evolved the ecology of the Earth, these organisms are being explored for potential applications in outer space programs[^{54,55}]. Therefore, in the long term this material presents possibilities for applications developing extra-terrestrial human habitations.

However, to apply this this material, its complex behaviour must be programmed for specific in-situ contexts. The preliminary tests conducted in this research show that the phototactic behaviour of the cyanobacteria can be further explored to extrinsically program the biomineralization patterns and metabolic impact of the material on the environment. Cyanobacteria are also known to respond to chemotactic signals such as CO₂, O₂ and HCO₃-ion concentrations, which can be used a material programming parameter as well.

### REFERENCES

1. Nguyen, P. Q., Courchesne, N. M. D., Duraj-Thatte, A., Praveschotinunt, P. & Joshi, N. S. Engineered Living Materials: Prospects and Challenges for Using Biological Systems to Direct the Assembly of Smart Materials. Adv. Mater. 30, 1-34 (2018).
2. Chen, A. Y., Zhong, C. & Lu, T. K. Engineering living functional materials. ACS Synth. Biol. 4, 8-11 (2015).
3. Srubar, W. V. Engineered Living Materials: Taxonomies and Emerging Trends. Trends Biotechnol. 1-10 (2020) doi: 10.1016/j.tibtech.2020.10.009.
4. Appiah, C. et al. Living Materials Herald a New Era in Soft Robotics. Adv. Mater. 31, (2019).
5. Bird, L. J. et al. Engineered living conductive biofilms as functional materials. MRS Commun. 9, 505-517 (2019).
6. de Belie N, Gruyaert E & Tabbaa A et al. A Review of Self-Healing Concrete for Damage Management of Structures. Advanced Materials Interfaces. 1-28 (2018).
7. Qian, Z. G., Pan, F. & Xia, X. X. Synthetic biology for protein-based materials. Curr. Opin. Biotechnol. 65, 197-204 (2020).
8. Gilbert, C. & Ellis, T. Biological Engineered Living Materials: Growing Functional Materials with Genetically Programmable Properties. ACS Synth. Biol. (2019) doi: 10.1021/acssynbio.8b00423.
9. Gilbert, C. et al. functionalities grown from engineered microbial. Nat. Mater. doi: 10.1038/s41563-020-00857-5.
10. Huang, J. et al. Programmable and printable Bacillus subtilis biofilms as engineered living materials. Nat. Chem. Biol. 15, 34-41 (2019).
11. Guo, S. et al. Engineered Living Materials Based on Adhesin-Mediated Trapping of Programmable Cells. ACS Synth. Biol. 9, 475-485 (2020).
12. Cachat, E., Liu, W. & Davies, J. A. Synthetic self-patterning and morphogenesis in mammalian cells: a proof-of-concept step towards synthetic tissue development. Engineering Biology vol. 1 71-76 (2017).
13. Ouyang, L., Armstrong, J. P. K., Salmeron-Sanchez, M. & Stevens, M. M. Assembling Living Building Blocks to Engineer Complex Tissues. Adv. Funct. Mater. 30, 1-22 (2020).
14. Başaran Bundur, Z., Bae, S., Kirisits, M. J. & Ferron, R. D. Biomineralization in Self-Healing Cement-Based Materials: Investigating the Temporal Evolution of Microbial Metabolic State and Material Porosity. J. Mater. Civ. Eng. 29, 04017079 (2017).
15. Gómez-Tatay, L. & Hernández-Andreu, J. M. Biosafety and biosecurity in Synthetic Biology: A review. Crit. Rev. Environ. Sci. Technol. 49, 1587-1621 (2019).
16. Zhou, K., Qiao, K., Edgar, S. & Stephanopoulos, G. Distributing a metabolic pathway among a microbial consortium enhances production of natural products. Nat. Biotechnol. 33, 377-383 (2015).
17. Gorochowski, T. E. et al. Toward Engineering Biosystems With Emergent Collective Functions. Front. Bioeng. Biotechnol. 8, 1-9 (2020).
18. Suosaari, E. P. et al. New multi-scale perspectives on the stromatolites of Shark Bay, Western Australia. Sci. Rep. 6, 1-13 (2016).
19. Dupraz, C. et al. Processes of carbonate precipitation in modern microbial mats. Earth-Science Reviews (2009) doi: 10.1016/j.earscirev.2008.10.005.
20. Arp, G., Reimer, A. & Reitner, J. Photosynthesis-induced biofilm calcification and calcium concentrations in phanerozoic oceans. Science (80-.). 292, 1701-1704 (2001).
21. Uyeda, J. C., Harmon, L. J. & Blank, C. E. A comprehensive study of cyanobacterial morphological and ecological evolutionary dynamics through deep geologic time. PLOS One 11, 1-32 (2016).
22. Govindjee & Shevela, D. Adventures with cyanobacteria: A personal perspective. Front. Plant Sci. 2, 1-17 (2011).
23. Kamennaya, N. A., Ajo-Franklin, C. M., Northen, T. & Jansson, C. Cyanobacteria as biocatalysts for carbonate mineralization. Minerals 2, 338-364 (2012).
24. Kupriyanova, E. V. & Pronina, N. A. Carbonic anhydrase: Enzyme that has transformed the biosphere. Russ. J. Plant Physiol. 58, 197-209 (2011).
25. Li, T., Sharp, C. E., Ataeian, M., Strous, M. & De Beer, D. Role of extracellular carbonic anhydrase in dissolved inorganic carbon uptake in alkaliphilic phototrophic biofilm. Front. Microbiol. 9, 1-12 (2018).
26. Umar, M., Kassim, K. A. & Ping Chiet, K. T. Biological process of soil improvement in civil engineering: A review. J. Rock Mech. Geotech. Eng. 8, 767-774 (2016).
27. De Muynck, W., De Belie, N. & Verstraete, W. Microbial carbonate precipitation in construction materials: A review. Ecol. Eng. 36, 118-136 (2010).
28. Heveran, C. M. et al. Biomineralization and Successive Regeneration of Engineered Living Building Materials. Matter 2, 481-494 (2020).
29. Hoiczyk, E. Gliding motility in cyanobacteria: Observations and possible explanations. Arch. Microbiol. 174, 11-17 (2000).
30. Babu, B. & Wu, J. T. Production of natural butylated hydroxytoluene as an antioxidant by freshwater phytoplankton. J. Phycol. 44, 1447-1454 (2008).
31. Meyer, G. E. & Neto, J. C. Verification of color vegetation indices for automated crop imaging applications. Comput. Electron. Agric. 63, 282-293 (2008).
32. Kaplan, A., Schwarz, R., Lieman-Hurwitz, J. & Reinhold, L. Physiological and molecular aspects of the inorganic carbon-concentrating mechanism in cyanobacteria. Plant Physiol. 97, 851-855 (1991).
33. Stadnichuk, I. N., Krasilnikov, P. M. & Zlenko, D. V. Cyanobacterial phycobilisomes and phycobiliproteins. Microbiol. (Russian Fed. 84, 101-111 (2015).
34. Singh, N. K., Sonani, R. R., Prasad Rastogi, R. & Madamwar, D. The phycobilisomes: An early requisite for efficient photosynthesis in cyanobacteria. EXCLI J. 14, 268-289 (2015).
35. Chen, W., Shi, Y. Q. & Xuan, G. Identifying computer graphics using HSV color model and statistical moments of characteristic functions. Proc. 2007 IEEE Int. Conf. Multimed. Expo, ICME 2007 1123-1126 (2007) doi: 10.1109/icme.2007.4284852.
36. Schwarz, M. W., Cowan, W. B. & Beatty, J. C. An Experimental Comparison of RGB, YIQ, LAB, HSV, and Opponent Color Models. ACM Trans. Graph. 6, 123-158 (1987).
37. Weiner, S. An Overview of Biomineralization Processes and the Problem of the Vital Effect. Rev. Mineral. Geochemistry 54, 1-29 (2003).
38. Yue, S., He, H., Li, B. & Hou, T. Hydrogel as a biomaterial for bone tissue engineering: A review. Nanomaterials 10, 1-25 (2020).
39. Burg, K. J. L., Porter, S. & Kellam, J. F. Biomaterial developments for bone tissue engineering. Biomaterials 21, 2347-2359 (2000).
40. Evans, J. S. Composite materials design: Biomineralization proteins and the guided assembly and organization of biomineral nanoparticles. Materials (Basel). 12, (2019).
41. Asenath-Smith, E., Li, H., Keene, E. C., Seh, Z. W. & Estroff, L. A. Crystal growth of calcium carbonate in hydrogels as a model of biomineralization. Adv. Funct. Mater. 22, 2891-2914 (2012).
42. Nindiyasari, F. et al. Effect of hydrogel matrices on calcite crystal growth morphology, aggregate formation, and co-orientation in biomimetic experiments and biomineralization environments. Cryst. Growth Des. 15, 2667-2685 (2015).
43. Nishinari, K., Hofmann, K. E., Moritaka, H., Kohyama, K. & Nishinari, N. Gel-sol transition of methylcellulose. Macromol. Chem. Phys. 198, 1217-1226 (1997).
44. Li, L. et al. Gel network structure of methylcellulose in water. Langmuir 17, 8062-8068 (2001).
45. Liang, H. F. et al. Novel method using a temperature-sensitive polymer (methylcellulose) to thermally gel aqueous alginate as a pH-sensitive hydrogel. Biomacromolecules 5, 1917-1925 (2004).
46. Li, H., Tan, Y. J., Leong, K. F. & Li, L. 3D Bioprinting of Highly Thixotropic Alginate/Methylcellulose Hydrogel with Strong Interface Bonding. ACS Appl. Mater. Interfaces 9, 20086-20097 (2017).
47. Ng, K. C. et al. Experimental investigation of the silica gel-water adsorption isotherm characteristics. Appl. Therm. Eng. 21, 1631-1642 (2001).
48. Bareschino, P., Pepe, F., Roselli, C., Sasso, M. & Tariello, F. Desiccant-based air handling unit alternatively equipped with three hygroscopic materials and driven by solar energy. Energies 12, (2019).
49. Ciriminna, R. et al. The sol-gel route to advanced silica-based materials and recent applications. Chemical Reviews vol. 113 6592-6620 (2013).
50. Adams, D. G. Heterocyst formation in cyanobacteria. Curr. Opin. Microbiol. 3, 618-624 (2000).
51. Zhang, C. C., Laurent, S., Sakr, S., Peng, L. & Bédu, S. Heterocyst differentiation and pattern formation in cyanobacteria: A chorus of signals. Mol. Microbiol. 59, 367-375 (2006).
52. Herrero, A., Stavans, J. & Flores, E. The multicellular nature of filamentous heterocyst-forming cyanobacteria. FEMS Microbiol. Rev. 40, 831-854 (2016).
53. Arioğlu Akan, M. Ö., Dhavale, D. G. & Sarkis, J. Greenhouse gas emissions in the construction industry: An analysis and evaluation of a concrete supply chain. J. Clean. Prod. 167, 1195-1207 (2017).
54. Cockell, C. S. Synthetic geomicrobiology: Engineering microbe-mineral interactions for space exploration and settlement. Int. J. Astrobiol. 10, 315-324 (2011).
55. Dade-Robertson, M., Ramirez-Figueroa, C. & Zhang, M. Radical vernacular: Bacterial architecture on Mars. JBIS—J. Br. Interplanet. Soc. 67, 346-353 (2014).

### Example 2

### Biomineralization and Structural Performance:

With reference to FIG. 19, Fourier transform infrared spectroscopy was performed on four samples of ELM and compared with three control samples to determine the precipitation of calcium carbonate. It is known that the range of absorbance of carbonate bonds in Calcite minerals is 1410-1490 cm⁻¹[²]. And it can be seen that all the four ELM samples had about 5% higher absorbance of infrared spectrum at 1450 cm⁻¹ in contrast to the control samples. This indicates the cyanobacteria catalysed biomineralization in the ELM samples.

With reference to FIG. 20 and Table 1, to evaluate the structural performance, six samples of the ELM of size 5 cm dia and 1 cm thickness were subjected to compression force until failure in a universal compression testing machine and compared to six abiotic control samples. It can be seen from the preliminary data that the ELM samples had 2% higher compression capacity in terms of Mega Pascals than the abiotic ones. This could be co-related with 5% more calcium carbonate seen in the previous test. However, it was noticed that samples with aggregate size 3-5 mm were more brittle with a generally lower failure value than the samples with aggregate size of 1 mm. At the same time the smaller 1 mm aggregate size samples were more malleable than the larger aggregate samples. There further tests need to be conducted with respect to standard testing protocols of plastic bodies and other properties like deformation and yield stress need to be evaluated.

TABLE 1
Data log of compression test conditions and results
Biotic samples - Cylinders

| | | | | Initial | Final | | | | | | | |
|---|---|---|---|---|---|---|---|---|---|---|---|---|
| | Aggregate | | Biofilm | radius | radius | Surface | Load/ | Failure | Max | Failure | Failure | Compressive |
| Sl no. | size | | color | [mm] | [mm] | area | sec | detection | load | load | newtons | strength |
| 1 | <1 | mm | Green | 50 | 55 | 7853 | 20 kg | 5% | 5000 kg | 5000 | 50,000 | 6.366993506 |
| 2 | <1 | mm | Blue | 50 | 58 | 7853 | 20 kg | 5% | 5000 kg | 5000 | 50,000 | 6.366993506 |
| 3 | <1 | mm | Yellow | 50 | 55 | 7853 | 20 kg | 5% | 5000 kg | 5000 | 50,000 | 6.366993506 |
| | | | | | | | | | | | Average | 6.366993506 |
| 4 | 3-5 | mm | Blue | 50 | 50 | 7853 | 20 kg | 5% | 5000 kg | 316.54 | 3165 | 0.403030689 |
| 5 | 3-5 | mm | Blue- | 50 | 50 | 7853 | 20 kg | 5% | 5000 kg | 413.53 | 3729 | 0.474850376 |
| | | | green | | | | | | | | | |
| 6 | 3-5 | mm | Green | 50 | 55 | 7853 | 20 kg | 5% | 5000 kg | 361.51 | 3,615 | 0.46033363 |
| | | | | | | | | | | | Average | 0.446071565 |

Ablotic controls - Cylinders

| | Aggregate | | Biofilm | Initial | Final | Surface | Load/ | Failure | Max | Failure | Failure | Compressive |
|---|---|---|---|---|---|---|---|---|---|---|---|---|
| Sl no. | size | | color | radius | radius | area | sec | detection | load | load | newtons | strength |
| 1 | <1 | mm | None | 50 | 55 | 7853 | 20 kg | 5% | 5000 kg | 4354 | 43,540 | 5.544377945 |
| 2 | <1 | mm | None | 50 | 50 | 7853 | 20 kg | 5% | 5000 kg | 313.01 | 3130 | 0.398573793 |
| 3 | <1 | mm | None | 50 | 52 | 7853 | 20 kg | 5% | 5000 kg | 5000 | 50,000 | 6.366993506 |
| | | | | | | | | | | | Average | 4.103315081 |
| 4 | 3-5 | mm | None | 50 | 50 | 7853 | 20 kg | 5% | 5000 kg | 331.53 | 3315 | 0.422131669 |
| 5 | 3-5 | mm | None | 50 | 50 | 7853 | 20 kg | 5% | 5000 kg | 309.49 | 3094 | 0.393989558 |
| 6 | 3-5 | mm | None | 50 | 50 | 7853 | 20 kg | 5% | 5000 kg | 328.42 | 3284 | 0.418184133 |
| | | | | | | | | | | | Average | 0.41143512 |

### Photosynthesis and CO₂ Sequestration (FIGS. 31-36):

The CO₂ sequestration by the ELM was studied under both phases-during growth and post curing applied phase.

To determine the CO₂ absorption during growth phase, a large-scale sample of size 60 cm×35 cm×3.5 cm, weighing 4 kgs was casted in a transparent casing box. The lid of the box was sealed airtight, and a KKmoon high sensitivity CO₂ sensor was placed within it. The data of CO₂ PPM, humidity and temperature were recorded over 333 hours of growth of the ELM. It was observed that although there is 12-hour cycle of CO₂ levels relating to the circadian rhythm of the cyanobacteria[³], the regression trend graph shows an overall decline of 1000 PPM of CO₂ inside the box during the growth phase.

To study the CO₂ absorption after the post curing phase, small 9 mm dia samples were prepared. A set-up box was set up such that the material can be inserted as a partition. Two Arduino CO₂ MQ135 sensors boards were placed, one to test the CO₂ levels of the air before passing through the ELM and one after passing through the ELM. Real time data was collected every two seconds for two and half hours. From the data below it could be observed that there was a constant absorption rate of 0.6 PPM per min with an average absorption of 29 PPM over the test period across the ELM samples.

### Cellular Viability and Regeneration Capacity:

To test the viability of the cyanobacteria within the ELM material over time, two samples were taken which were cured and kept under ambient condition without any additional support. Sample 1 was 60 days old and sample 2 was 180 days old. Both samples were then supplement in fresh BG11 media solution and growth activity of the cyanobacteria was recorded over 2 months. It could be seen that although there was a longer lag in growth activation for the 180 days old sample, both samples exhibited significant biofilm growth after 2 months under fresh media. This confirms the viability of the cells and that the material is regenerative. (FIG. 26).

### BIBLIOGRAPHY

1. Van Vlierberghe, S., Dubruel, P. & Schacht, E. Biopolymer-based hydrogels as scaffolds for tissue engineering applications: A review. Biomacromolecules 12, 1387-1408 (2011).
2. Nandiyanto, A. B. D., Oktiani, R. & Ragadhita, R. How to read and interpret ftir spectroscope of organic material. Indones. J. Sci. Technol. 4, 97-118 (2019).
3. Liu, Y. et al. Circadian orchestration of gene expression in cyanobacteria. Genes Dev. 9, 1469-1478 (1995).
4. Adams, D. G. How do cyanobacteria glide? 28, 131-133.
5. ITIS report. ITIS Report, Oscillatoriaceae, Taxonomic serial number: 862. https://www.itis.gov/servlet/SingleRpt/SingleRpt?search_topic=TSN&search_value=862 #null.
6. Li, H., Tan, Y. J., Leong, K. F. & Li, L. 3D Bioprinting of Highly Thixotropic Alginate/Methylcellulose Hydrogel with Strong Interface Bonding. ACS Appl. Mater. Interfaces 9, 20086-20097 (2017).
7. Zuidema, J. M., Rivet, C. J., Gilbert, R. J. & Morrison, F. A. A protocol for rheological characterization of hydrogels for tissue engineering strategies. J. Biomed. Mater. Res.—Part B Appl. Biomater. 102, 1063-1073 (2014).

1. A method of producing a biomineralized material comprising calcium carbonate-bonded aggregate, the method comprising:
culturing a photosynthetic microorganism in a hydrogel matrix, wherein the microorganism releases extracellular carbonic anhydrase into the hydrogel matrix, and
wherein the hydrogel matrix comprises:
i) a hydrogel;
ii) an aggregate material;
iii) growth media; and
iv) calcium chloride (CaCl₂)),
wherein the extracellular carbonic anhydrase converts the calcium chloride to calcium carbonate precipitate thereby bonding the aggregate material to form the biomineralized material.

2. The method according to claim 1, wherein the aggregate material is translucent.

3. The method according to claim 1, wherein the aggregate comprises or consists of a particulate material having a micro-porous surface.

4. The method according to claim 1, wherein the aggregate comprises or consists of a material selected from amorphous silica (SiO₂), crystalline quartz silica, glass particles, transparent ceramics, polyacrylate and glass fibres; or combinations thereof.

5. The method according to claim 1, wherein the aggregate material comprises amorphous silica (SiO₂).

6. The method according to claim 1, wherein the aggregate material further comprises activated carbon pellets and/or zeolite.

7. The method according to claim 1, wherein the aggregate material comprises or consists of activated carbon pellets and amorphous silica (SiO₂) in a ratio of between 1:2 and 1:4.

8. The method according to claim 1, wherein the microorganism exhibits gliding motility, and phototactic response and/or chemotactic response.

9. The method according to claim 1, wherein the microorganism comprises filamentous cyanobacteria.

10. The method according to claim 1, wherein the microorganism comprises filamentous cyanobacteria that is capable of forming dormant cells and/or survive in terrestrial conditions.

11. The method according to claim 1, wherein the microorganism is of the family Oscillatoriaceae; or wherein the microorganism comprises or consists of Oscillatoria animalis.

12. (canceled)

13. The method according to claim 1, wherein the hydrogel is thixotropic.

14. The method according to claim 1, wherein the hydrogel comprises methylcellulose.

15. The method according to claim 1, wherein the hydrogel comprises or consist of methylcellulose and one or more of sodium alginate, agar and carrageenan.

16. The method according to claim 15, wherein the methylcellulose and one or more of sodium alginate, agar and carrageenan are in a ratio of between about 15:1 and 3:1 methylcellulose relative to the total of the other gel components.

17. The method according to claim 1, wherein a surface of the hydrogel matrix is shaped to provide one or more of ridges, flanges, projections, indentations, grooves, channels, bumps, and undulations.

18. The method according to claim 1, wherein the calcium chloride is provided at a concentration of between about 0.01M and about 1M.

19. A composition for producing a biomineralized material comprising calcium carbonate-bonded aggregate, the composition comprising a hydrogel matrix, wherein the hydrogel matrix comprises:
i) a hydrogel;
ii) an aggregate material;
iii) growth media;
iv) calcium chloride (CaCl₂)); and
v) a microorganism, wherein the microorganism is capable of expressing and releasing extracellular carbonic anhydrase into the hydrogel matrix.

20. A biomineralized material comprising:
i) a hydrogel or a biogenic mineral of a dried hydrogel
ii) calcium carbonate-bonded aggregate material;
iii) a filamentous cyanobacteria or spore-like cells thereof capable of forming reproductive cells of the filamentous cyanobacteria, wherein the filamentous cyanobacteria is capable of expressing extracellular carbonic anhydrase.

21. (canceled)

22. A biomineralized material produced by the method in accordance with claim 1.
